(19) United States
(12) Patent Application Publication
(10) Pub. No.: US 2006/0088508 A1
(43) Pub. Date: Apr. 27, 2006

(54) Method for obtaining characterized muscle-derived cell populations and uses

(75) Inventors: Jean-Thomas Vilquin, Chelles (FR); Jean-Pierre Marolleau, Le Perreux (FR); Jacques Tremblay, Quebec (CA); Isabelle Robert, Saint-Quen (FR); Brigitte Ternaux, Villepreux (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignees: ASSISTANCE PUBLIQUE - HOSPITAUX DE PARIS, Paris (FR); INSTITUT NATIONAL DE LA SANTE ET DE LA RECHERCHE MEDICALE, Paris Cedex (FR); ASSOCIATION FRANCAISE CONTRE LES MYOPATHIES, Paris (FR)

(21) Appl. No.: 11/207,795

(22) Filed: Aug. 22, 2005

Related U.S. Application Data
(63) Continuation of application No. 10/314,257, filed on Dec. 9, 2002, now abandoned.
Application No. 10/314,257 is a continuation of application No. PCT/FR01/01768, filed on Jun. 7, 2001.

(30) Foreign Application Priority Data
Jun. 7, 2000 (FR) 00/07304

(51) Int. Cl. A01N 1/02; C12N 5/08; A61K 35/34

(52) U.S. Cl. 424/93.7; 435/2; 435/366

(57) ABSTRACT

A method for obtaining cell populations derived from the muscular tissue and their use for preparing cell therapy products includes culturing cells previously removed by biopsy from skeletal muscular tissues, identifying the different types of cells present at different stages of culture, selecting the culture stage on the basis of the required cell population and collecting the selected culture stage for preparing a cell therapy product. The invention also concerns cell populations derived from muscular tissue obtained by implementing the method whereof the dominant cell type is CD34+, CD15+ or CD56+ or Class 1+ HLA, or comprises a doubly negative CD56−/CD15− cell type or may comprise more minority CD10+, Stro-1+ and CD117+ cell types.

The present invention concerns cell populations derived from muscle tissue and their use in the preparation of cell therapy products. More specifically, the invention concerns a method of obtaining cell populations and their use for reconstituting the hematological and immunological system, and the bone, adipose, cartilage, muscle or vascular tissues.

Cell therapy is a method With promising potential for the treatment of many diseases. The principle of cell therapy is based on the possibility of reconstituting damaged tissue or of restoring a biological function that has been lost or impaired within a tissue, from specific cells cultured ex vivo and transplanted onto the sick tissue. Another interest of cell therapy is that the transplanted cells can be used as a platform for delivering a biologically active product, if necessary after genetic modification of the cells before transplantation. Many trials of cell therapy have been reported using primary cultures of different cell types. We could mention the transplantations of neuronal cells-carried out to treat Huntingdon's chorea (1) or Parkinson's disease (2), transplantations of islet of Langerhans cells to treat diabetes (3) or transplantations of myoblastic cells carried out to treat Duchenne's muscular dystrophy (4,5,6,7) or, after genetic modification of the cells, for the treatment of dwarfism (8), hemophilia (9) and Parkinson's disease (10).

Skeletal muscle is regenerated by the satellite cells, which are mononucleate myogenic cells located under the basal layer of the muscle fibers. Following a lesion, these cells quit a quiescent state and embark on a phase of active proliferation and are known subsequently as myoblasts. Subsequently, the myoblasts fuse to form myotubes. There have been attempts in man to transplant myoblasts to treat Duchenne's muscular dystrophy and Becker's muscular dystrophy (4,6,7,11). The functional effect of the transplantations described in these studies remains limited, but no side effect has been reported in terms of infection or carcinogenesis.

Furthermore, the use of myoblast cells to treat heart disease, and in particular, to treat post-ischemic heart failure, has been envisaged. Indeed, unlike muscle tissue, the myocardial tissues are devoid of stem cells able to produce cardiomyocytes and regenerate the tissues. At the present time, the most radical treatment available for post-ischemic heart failure is still heart transplantation. However, the shortage of transplants available limits this therapeutic use. The transplantation of cells derived from muscle tissue into the heart muscle has therefore been envisaged as an alternative to heart transplantation. Transplants of myoblasts into the heart muscle have been carried out in rat, rabbit and dog studies (12, 13, 14). The results of these studies have demonstrated that such transplants are feasible and have some functional effect. In studies of a model of iatrogenic heart failure induced in mice, transplants of fetal cardiomyocytes have been shown to have some functional advantage. However, with a view to clinical applications, using fetal cells poses a variety of ethical and immunological problems, and that of the supply of cells. Using a population of myoblastic cells derived from skeletal muscle is therefore a particularly promising alternative for the preparation of cell therapy products for treating post-ischemic heart failure and indeed for the treatment of various heart diseases.

One of the most important cell types found in muscle tissue is that of the satellite cells, which are precursors of myoblasts. This is the cell type that has been used in the various clinical studies. However, the muscle tissue also contains other types of cell. In particular, some cells of muscular origin could also be used to reconstitute the hematopoietic potential (15, 16). An in-vitro study has also demonstrated that human muscle contains progenitors that could differentiate in the long term to form cartilage or bone tissues (17). Examples of media suitable for obtaining differentiation into adipose, cartilage or bone tissue have been described for mesenchymatous stem cells (22).

In consequence, in view of the differentiation potential of cells of muscular origin, using these cells for cell therapy looks promising for the treatment of many lesions affecting the tissues of the hematological and immunological system, and bone, adipose, cartilage, muscular or vascular tissues.

One major difficulty associated with cell therapy remains that of obtaining a population of cells that is sufficiently large and uniform, and has a degree of differentiation appropriate for the desired effect.

Methods of preparing myoblastic cells and their use for cell therapy have been described in reports of state of the art techniques (4, 18, 19, 20, 21, 25, 26, 27). Most of these methods include:
a step of removing muscle tissues by biopsy
a mincing step
a step of dissociating the muscle fibers by an enzymatic effect,
a step of separating the initial cells by filtration
a step of selecting the myoblastic cells by cloning or cell sorting.

In order to obtain a sufficiently dense and rich population, or even populations consisting entirely of myoblastic cells, it has been suggested that myoblastic cells could be selected on the basis of the expression of specific markers. Thus, the selection of myoblastic cells can be achieved by cloning the cells and subsequently characterizing the clones obtained by cytofluorimetry, followed by selection of the skeletal muscle cells expressing the CD56 antigen (7). A direct method of sorting the myoblastic cells expressing the CD56 antigen by flow cytofluorimetry and its advantages for obtaining a pure culture of myoblasts are also described in state of the art techniques (21).

The cells that are retained are then cultured in a modified culture medium, specially adapted for the culture of myoblasts (23).

The present invention results from the observation that cells derived from skeletal muscle can potentially regenerate numerous tissues depending on their degree of differentiation. The invention proposed therefore makes it possible to provide clearly characterized cell populations or muscular origin, which are adapted and specially prepared for their intended use in cell therapy.

The present invention provides a method for obtaining a cell population consisting of a dominant cell type from a muscle tissue biopsy, for the preparation of a cell therapy suitable for human use, the said method including the following steps:
a) taking and mincing a muscle biopsy specimen
b) enzymatic dissociation of the muscular fibers and cells, and separating the individual cells by filtration
c) culture of the cells of muscular origin thus obtained in an adhering cell culture reactor in the presence of a growth and/or differentiation medium, followed if appropriate by one or more expansion phase(s),
d) identification of the cell types present at various stages of the culture by analysis of specific cell markers,
e) selection of the stage of culture during which the target cell type constitutes a dominant proportion of the cell population
f) harvesting of a population of cells at the stage of culture selected in e),
g) if necessary, freezing of the cells collected at the step selected for the preparation of the cell therapy product*.

Step d) is optional to the extent that if the method is used several times under the same conditions, the investigator knows which cell types are present at various stages in the culture and their relative proportions without having to repeat the identification step.

It has indeed been found that the identification step (d) leads to virtually the same results when the same method is repeated.

In a particular form of the invention, the method also involves the use of depletion and enrichment techniques before the culture step c) or before expansion in order to alter the proportions of the various cell types.

The terms “cell population” and “population of cells” both indicate any population of cells which is not pure; usually containing a dominant cell type combined with one or more minority cell types. The dominant cell type is the cell type present in the highest proportion in the cell population. A dominant cell type is preferably the cell type constituting more than 50% of the cell population. A cell therapy product suitable for human administration consists of an isotonic solution in which the cells are resuspended. This solution must be devoid of the toxic constituents present in the freezing media, such as DMSO.

The invention results in, particular from the observation that the method can be used to obtain a cell population with a composition appropriate for the intended therapeutic effect. In particular, it makes it possible to obtain a population of cells in which the dominant cell type expresses the CD56+ marker and the class I-HLA marker, without preliminary sorting or positive selection for cells expressing the CD56+ marker.

The muscle biopsy is generally carried out by taking specimen cubes with sides measuring 2 to 4 cm. According to requirements, specimens of between 0.05 grams up to several tens of grams can be taken. One of the advantages of the method is that it can be used to obtain a very large number of cells of a cell type present in a dominant proportion, ranging from a few thousand to several billion, depending on the target cell type, the number of expansions performed and the time allowed for each passage. By way of example, the method can be used to produce up to several hundred million cells expressing the CD56 antigen within a period of two to three weeks. As the method permits numerous expansion phases, it can theoretically be used to obtain at least 100 billion cells expressing the CD56+ antigen and class-1 HLA antigen after 8 or 9 expansions. The muscle tissue used is skeletal muscle tissue, preferably taken from an adult, young adult, adolescent or child. In one form of the invention, the muscle tissue taken is a fetal skeletal muscle tissue. The cells can notably be obtained from the vastus lateralis, vastus medialis, biceps, quadriceps, tibialis, gastrocnemius, peroneus, deltoid, lassimus dorsi, sternocleidomastoid, intercostal, homohyoid, rectus abdominis or psoas muscle.

The mincing process consists of cutting the biopsy specimen into sections, preferably measuring less than 0.5 mm, which are placed in an appropriate culture medium. Mincing is an essential step to allow effective subsequent enzymatic dissociation department. Mincing can be carried out manually using fine scissors. However, unexpectedly, it has been discovered that when the mincing step is carried out with assistance, using homogenizers with electrically or mechanically driven blades, the method of the invention in which the culture medium is appropriate for the differentiation into myoblasts, yields a population with a particularly high percentage of cells expressing the CD56 antigen. One example of a homogenizer of this type that can be used is the Medimachine® homogenizer (distributed by Becton-Dickinson).

Consequently, in one form, the method of the invention is characterized by the fact that the mincing step is assisted using homogenizers with mechanical or electrical blades.

A muscle tissue consists of muscle fibers. The satellite cells are located under the basal layer of the muscle fibers. The step in which the muscle fibers are dissociated and the satellite cells are detatched is therefore an essential step in their isolation.

The dissociation step consists of using enzymes to digest the extracellular matrix, this can be completed by mechanical dissociation and aspirating and ejecting the suspension using a pipette.

The choice of enzymes and the concentrations used to separate the muscle fibers and the satellite cells from the minced tissue is guided by a determination of their enzymatic efficacy, the target criteria are the lowest possible concentration of enzyme and the shortest possible incubation time for a similar efficacy. The yield in cells obtained after filtration depends in part on the quality of the enzymatic dissociation step. Digestive enzymes that are suitable for use alone or in combination in the method of the invention are, for example:
all the collagenases, including the partially purified IA, S and H types, and the purified form marketed by Roche-Boehringer, under the name Liberase,
the trypsins, of any origin, in solution in buffers with or without EDTA,
dispases (also known as proteases),
pronase,
elastases,
or hyaluroindases.

The dissociation step is preferably carried out in two stages: a first incubation in the presence of collagenase and a second incubation in the presence of trypsin. When Liberase is used, the most effective concentrations used in the mince are between 0.05 and 2 mg/ml. The incubation time at 37° C. for these concentrations being selected in this case ranges from 15 minutes to 2 hours. The activity of the dissociation enzymes is preferably neutralized after dissociating or detaching the cell layer in order to avoid damaging the cells.

After neutralizing the enzymatic activity, by adding, for example, fetal calf serum, autologous human serum or allogogous human serum from a compatible group or an inhibitor of the enzyme activity, the digestion product is filtered over a strainer, under gravity, in order to eliminate any undissociated fibers and to collect the cells detached from the muscle fibers. The filtration step should preferably be performed in two stages: one filtration step over a 100-1 μm strainer and then a second step using a 40-μm strainer.

The cells collected after filtration are transferred into a culture reactor in the presence of a medium with a composition permitting their growth and/or differentiation. The composition of the medium is chosen on the basis of the dominant cell type wanted at the end of the culture. At this stage, part of the preparation can be frozen. This may be part of the initial preparation or a sub-population obtained after an enrichment or depletion step. The culture media used contain one or more growth and/or differentiation factors which are intended to steer the progenitor cells towards a specific differentiation pathway and to make them proliferate. As examples of growth factors, we could list the fibroblast growth factors, bFGF, aFGF, FGF6, the hepatocyte growth factor HGF/SF, the epidermis growth factor, EGF, and the various factors identified such as IGF-1, PDGF, LIF, VEGF, SCF, TGFb, TNFa, IL-6, IL-15, NGF, neuregulin, thrombopoietin and growth hormone. They can be combined with various hormones or active molecules which can be included in the composition of media, such as the glucocorticosteroids (natural or semi-synthetic, i.e. hydrocortisone, dexamethasone, prednisolone or triamcinolone), progestagens and their derivatives (progesterone), estrogens and their derivatives (estradiol), androgens and their derivatives (testosterone), the mineralocorticosteroids and their derivatives (aldosterone), the hormones LH, LH-RH, FSH and TSH, the thyroid hormones T3 and T4, retinoic acid and its derivatives, calcitonin, the prostaglandins E2 and F2/alpha or parathyroid hormone.

Before being put in culture or during an expansion phase, the preparation can be subjected to enrichment or depletion. These operations are carried out by a specialist using the various methods available in state of the art techniques to carry out a selective sorting procedure. These techniques are based on identifying characteristic extracellular antigens of a given cell type by specific reagents. By way of example, the CD34 and CD56 antigens expressed on some of the cells present in a population of muscle cells can be used. The initial sorting of the total cell population according to whether they express these two antigens can be used to divide them into two groups. In particular, this can be used to separate the CD34+ type cells from the CD34− cell types. It has been shown that the CD34+ population generates a dominant CD15+; CD56− cell type which does not express desmin. The CD34+ population generates a dominant CD15+; CD15− cell type which expresses desmin. Occasionally, the CD34− population generates a CD56−, CD15− population. In particular, the method of the invention includes a CD34+ or CD34− cell depletion step, yielding a population consisting of a dominant CD15+ or CD56+ cell type respectively.

One way of performing the method of the invention therefore involves a process for obtaining a cell population in which a dominant cell type is the myoblastic cell type characterized by the fact that it includes a CD34+ cell depletion step before the cells are cultured or during one of the expansion phase.

The cells are then cultured in a reactor designed for the culture of adherent cells. In order to avoid the constraints of checking the speed and regularity of stirring and the uniformity of the preparations, the cuture reaction is preferably static. It should have a large culture area compared to conventional containers (Petri dishes, flasks), so that a large cell population can be harvested within a few days. An example of such a culture reactor is the tray culture system (single, double and/or multi-tray versions).

The culture system used in the method also makes it possible to sample the cells in a sterile fashion in order to take the samples necessary to identify the cell types present at the various stages of the culture, by analyzing the special markers. It makes it possible to empty the media, and to wash and detach the cells and finally to harvest them under sterile conditions.

Preferably, bags are used and specially designed tubes connect the bags to the reactor to permit the transfer of the media to another container or to harvest the cells. This system makes it possible to carry out a large number of operations in a closed system. Depending on the cell population wanted, the number of days in culture ranges from 0 to 45 days.

In addition, the culture can be continued by conventional expansion or perfusion methods for a period that may extend over several months.

One or several expansion phases can be used to increase the number of cells harvested. The expansion phases consist of a cell detachment step, washing the cells and returning them to culture on a larger surface area, the solutions and enzymes used to carry out these steps being familiar to the specialist.

In particular, in one preferred way of carrying out the method, using an appropriate culture medium for differentiating myoblasts, the method of the invention includes at least three cell expansion phases. Such a method makes it possible to multiply the number of cells without significantly altering the proportions of the cell types obtained at the end of the culture of each expansion.

A differentiation kinetic analysis is carried out in the method of the invention by identifying the cell types present in the cell populations obtained at the various stages of the culture. In the text which follows, the step in which the cell types present at various stages of the culture are identified is designated by the term “characterization of the cell populations”. This characterization is carried out using samples taken before the cells are cultured, during the culture and when the cells are harvested. The characterization of the cell populations can also use a culture of cells carried out in parallel on a smaller scale but under the same or equivalent conditions in terms of culture medium and how the expansion phases are conducted. The characterization involves adherent cells or the cells present in the supernatant. The different stages in the culture are counted in days from the day when the cells are put in the reactor to culture or when a degree of confluence of at least 20 to 50% of the cells is obtained. The purpose of characterizing cell populations is to identify all the cell types and their proportions as a function of the culture stage. In particular, it can be used to identify the dominant cell types as a function of the culture stage. The method according to the invention therefore provides a way to select a cell population in the light of the characteristics of the different cell types present in the population and of the objective of the intended cell therapy.

This characterization is carried out by an analysis of cell markers by flow cytofluorimetry or FACS after marking the surface antigens or of any specific antigen of the cell types to be analysed. In the present text, the term “cell markers” indicates any cell antigen that can provide information, either alone or in combination with other markers, about a cell type. Any cell marker can be used in the method, the choice of the markers used will depend on the choice of cell types to be characterized.

Any appropriate method can be used to characterize a cell antigen by the method of the invention. By way of example, and without being exclusive, we could mention Western Blot or immunocytochemistry. Any methods able to characterize the messenger RNA coding for the said antigens can be used in an equivalent fashion.

In one way of carrying out the invention, the cell markers analysed for identifying the cell types are specifically chosen from amongst the following markers: CD10, CD13, CD15, CD16, CD34, CD38, CD40, CD44, CD45, CD56, CD71, CD80, CD117 or the following structures CD138, Class-I HLA, class-II HLA, VLA3, VLA5, VLA6, ICAM-1, VCAM and desmin. Preferably, the markers CD10, CD13, CD15, CD34, CD44, CD56, CD117 and Class-I HLA and desmin are used. These markers are identified using specific monoclonal antibodies. Table 1, shown in the experimental section below, indicates for each of these markers, the cell types classically expressing the corresponding antigens. It should be noted that these cell markers were developed initially for characterizing the immuno-hematological system. One novel feature of the invention concerns using these markers to characterize cells of muscular origin at the various stages of the culture or of the expansion.

The method according to the invention is therefore implemented by means of an analysis of the cell markers not usually used in the characterization of the cells derived from muscle tissues. These cells markers are, for example, CD10, CD13, CD15, CD34, CD44, CD45, CD117 and Class-I HLA.

It has indeed been observed that implementing the method of the invention can be used to demonstrate a change over time in the dominant cell type present in the culture. In a surprising fashion, at the early stages of the culture of muscle tissue cells and in a culture medium suitable for myoblastic differentiation, the majority populations are those which express the markers for the progenitor cells of the bone marrow and the cells of the lymphoblastoid system. More precisely, it has been observed that on D0 and D1, most of the cells were of the CD34+/45− phenotype and the presence of a minority CD117+ cell type in the population of non-adhering cells. The development is then marked by a progressive increase in the proportion of CD15+ and/or CD56+ cells, and a fall in the CD34+ populations. Nevertheless, the dominant cell type remains CD34+ from D0 to D5. A progressive transition is observed from a population with a dominant CD34+ cell type to a dominant CD56+ cell type. In addition, the proportion of CD13+, CD44+, CD71+ cell types increases with time. The minority CD138+, Class-II HLA and CD38+ cell types are also observed. At the end of the culture, the dominant cell type is the CD56+ phenotype, in particular from the D5 stage until the end of the culture. The CD56+ cells also express CD10, CD13, CD44, desmin, Class-I HLA, CD44, VLA-3, VLA-5 and VLA-6. These markers characterize the myoblastic cells. A second preponderant cell type consists of cells expressing CD15, CD10, CD13 and Class-I HLA. A CD56−/CD15−/CD13+ population is also present in variable proportions, and constitutes a third preponderant cell type.

Within this population, some of the cells express desmin and others do not. The implementation of the method of the invention has thus made it possible to distinguish three cell types present in the cell populations, the proportions of which change considerably during culture: CD34+ cells, CD15+ cells and CD56+ cells.

In a preferred implementation of the method of the invention, the characterization of the cell populations consist of determining the respective percentages of the three cell types, CD34+, CD15+ and CD56+, at various steps in the culture.

In one form of the invention, a target cell type within a cell population is separated after selecting the culture stage during which the target cell type is in the qualitative or quantitative state sought, in particular, after selecting the culture stage during which the target cell type is dominant. The presence of the cells to be separated in a dominant proportion makes is easy to prepare them. The invention thus provides a process for obtaining a cell population with a high degree of purity. By a cell population with a high degree of purity is meant a population of cells in which the dominant cell type constitutes more than 50% of the cell population. It may be necessary to obtain a cell population with a high degree of purity for some uses as a cell therapy product. It is clear that a specialist could implement the various methods existing at the present state of the art to carry out selective sorting of the said cells. By way of example, let us mention the techniques of sorting by cloning, by flow cytofluorimetry or by immunoaffinity or immunomagnetic columns using specific antibodies of the cells concerned.

In another implementation, in contrast, the method of the invention is characterized by the fact that it does not have any steps involving the purification, positive selection or cloning of a specific cell type. By positive selection, should be understood any step making it possible to sort the cells on the basis of at least one distinctive characteristic and notably the expression of a cell marker. It has been shown, in particular, in contrast to the methods described in state of the art reports, to provide a cell therapy product consisting of a dominant myoblastic type, with no step involving the preferential selection of the myoblastic cell type. The absence of cloning, purification, or positive selection steps can considerably improve the final yield obtained at the end of the expansion procedures in terms of the numbers of cells obtained.

In the method according to the invention, after stopping the culture at the culture stage chosen, an aliquot of cells can be taken and cultured separately. The medium can be the same or have a different composition. The culture media are chosen in the light of the intended differentiation pathway. One example of a medium which can be used to differentiate the sample into myoblasts, endothelial cells, smooth muscle cells or myofibroblasts is MCDB medium 120, supplemented with fetal calf serum (23) which includes, in particular, a glucocorticosteroid, such as dexamethasone and bFGF. Another example of a preferred medium is modified MCDB 120 medium in which L-valine is replaced by D-valine. It has been observed that this change gives a medium, that is particularly selective for the differentiation of the cells to form myoblasts. Such a medium can be used with the method of the invention to yield a population containing a large number of cells, most of which are CD56+ or Class-I HLA type.

A medium for differentiation into adipose tissue contains in particular dexamethasone, isobutyl-methylxanthine and, in some cases, fetal calf serum, indomethacin and insulin. Differentiation is maintained in a medium containing 10% fetal calf serum and insulin. To obtain the differentiation into cartilage tissue, the cells are centrifuged to form micromasses and cultured in serum-free medium containing TGF-beta3. A medium for differentiation into bone tissue contains dexamethasone, beta-glycerophosphate, ascorbate and, in some cases, fetal calf serum.

The cells are harvested at the culture stage selected on the basis of the cell population that one is trying to obtain. Any non-adhering cells present in the supernatant and/or the adherent cells can be harvested by this method. The populations of cells present in the supernatant and those of adherent cells may have differing compositions. Consequently, the method of harvesting the cells will depend on the target cell population. Adherent cells are harvested by enzymatic dissociation of the cells and detachment of the cell layer by method known to the specialist. The non-adherent cells are harvested by aspiration.

The implementation of the above method including the establishment of a differentiation kinetic analysis from the cells derived from muscle tissue biopsies yields characterized cell populations for the preparation of a cell therapy product suitable for human administration. Consequently, the invention also concerns cell populations that can be obtained by the method of the invention.

It should be pointed out in particular that the invention also concerns populations of cells obtained by a similar method to the method of obtaining the cell populations described above, but which do not include a step identifying the cell types at different cell stages. Indeed, the step of identifying the cell types is necessary to chose the harvesting stage. Consequently, having implemented the method of the invention at least once, the specialist will know the best stage to harvest the cells depending on the target population, and can obtain the same types of populations by limiting the number of markers used to characterize the cell populations and the stages during which this characterization is performed. Thus, “populations of cells likely to be obtained” must therefore include a population of cells obtained by the method of the invention that may not necessarily include a cell population characterization stage according to the invention, such as that described above. The best stage for harvesting being identified by carrying out a differentiation kinetic analysis on a preliminary culture, carried out under the same conditions.

The various cell populations which can be obtained by the method of the invention, with or without an identification step are of different types, depending on the culture stage and culture medium chosen.

In particular, at the early stages of cell culture, the invention yields a cell population in which the dominant cell type expressed the CD34 marker. When only non-adherent cells are harvested at an early stage of the culture, the cell population also includes a minority cell type with the CD117+ phenotype.

In particular, it yields a cell population derived from the same muscle biopsy and consisting of 1.10⁵ to 1.10⁷ cells, 10 to 70% of which, and preferably more than 30%, of which are CD34+.

The invention also concerns the use of a cell population of muscular origin in which the dominant cell type expresses the CD34 marker, which is specific to pluripotent cells as a cell therapy product for reconstituting tissues of the hematological and immunological system or bone, adipose, cartilage, muscle or vascular tissues.

By implementing the method described above in which the medium is suitable for myoblast differentiation, the invention yields a cell population in which the dominant cell type expresses the CD15 marker.

The invention also concerns the use of a cell population in which the dominant cell type expresses the CD15 marker in the preparation of a cell therapy product for reconstituting skeletal, cardiac and visceral muscle tissues and vascular tissues.

Finally, by carrying out the method described above and using a medium suitable for myoblast differentiation, one preferred way of carrying out the invention yields a cell population in which the dominant cell type consists of myoblastic cells. The myoblastic cells are characterized by analysis of the expression of the CD56 marker. They are preferable characterized by analyzing the expression of the CD56 marker combined with an analysis of the CD10, CD13, CD44, Class-I HLA markers and desmin. Analysis of the expression of desmin, a specific intracellular protein of the cytoskeleton of myoblastic cells and differentiated muscle cells requires a suitable protocol for labeling and FACS analysis, described in the experimental section. The cell population also includes a CD56−/CD15− doubly negative population.

In particular, the invention yields a cell population derived from a single muscle biopsy and including 50×10⁶ to 800×10⁹ cells, preferably at least 500×10⁶ cells, of which at least 50% or better at least 60%, or preferably at least 70% are CD56+.

The invention also concerns a cell population in which the dominant cell type consists of myoblastic cells in the preparation of a cell therapy product for reconstituting skeletal, cardiac, and visceral muscle tissues and vascular tissues in human subjects.

In particular, the invention concerns the use of a cell population obtained according to the methods described above in the preparation of a cell therapy product for human use in the treatment of post-ischemic heart failure or for the repair of cardiac tissues. It also concerns the treatment of vascular disorders. Indeed, implementation of the method makes it possible to obtain a large number of cells rapidly and the cell populations obtained have the advantage of being homogeneous, and are therefore particularly suitable for the preparation of a cell therapy.

It also concerns the use of a cell population derived from a single muscle biopsy and containing 50×10⁶ to 800×10⁹ cells of which at least 50%, or preferably at least 60% are CD56+ or Class-I HLA in the preparation of a cell therapy product for the treatment in human subjects of post-ischemic cardiac failure or of heart diseases of genetic, viral, iatrogenic, infectious or parasitic origin.

The treatment of heart diseases consists in particular of injecting, by means of a needle, a population of cells in which the dominant type has the characteristics of myoblastic cells, obtained and prepared as a cell therapy product, directly into the myocardial tissues (12), or indirectly into the arterial bloodstream (24).

Preferably, at least 600×10⁶ cells derived from the same biopsy are injected.

Heart failure is now managed by treatment with angiotensin-converting enzyme inhibitors (ACEI). The experiments described below indicate that transplantating myoblastic cells in situ into the infarcted zone has a definite beneficial effect when this transplantation is carried out concomitantly with ACEI treatment.

The invention therefore also concerns the use of cell populations of muscular origin obtained by the methods described above as a transplant to enhance the pharmacological treatments of heart failure.

The experimental section of the present text describes the performance of transplantations of autologous cells of muscular origin in the rat, demonstrating the feasibility of this method. Indeed the results show that transplantation of these cells in the rat significantly improves the functional assessment parameters, thus demonstrating the feasibility, of such transplantations. They also shown that the improvement in myocardial function is related to the number of cells transplanted.

The experimental section also describes the results obtained in the rat, in, the combined use of autologous muscle cells and the pharmacological treatment of heart failure.

The invention also concerns the use of a population of cells obtained by the methods described above as a cell therapy product for the treatment of innate or acquired muscular dystrophies. In dystrophy patients, the transplantation of myoblasts can be used to restore the expression of dystrophin (6). It consists, for example, of using a needle to inject cells of muscular origin obtained by a method according to the invention directly into the skeletal muscle or into the general circulation (15).

It also concerns the use of a population of cells able to reconstitute the muscle, cartilage or bone tissues for the treatment of muscular and/or joint and/or osteotendinous lesions caused by trauma. The said population of cells is prepared as a cell therapy product and injected directly into the injured tissue or near by.

During the cell culture and expansion phases in the methods provided by the invention, a step may be carried out involving the genetic modification of the cells by transfection of a heterologous nucleic acid. The nucleic acid is chosen in order to permit the expression of a polypeptide or of a protein into the transfected cells. The transfected cells are then transplanted and make it possible to deliver a polypeptide or protein expressed from the heterologous nucleic acid, the said polypeptide or protein being a biologically active product. The invention thus concerns the use of a cell population as a cell therapy product to provide a platform for delivering a biologically active product.

The experimental section that follows illustrates, without restricting its scope, the implementation of the method according to the invention and its use. It involves three parts:

The first part describes examples of the implementation of the invention which, depending on the stage of culture chosen, to obtain cell populations in which the dominant cell type is CD34+, CD15+ or CS56+ (myoblastic) or CD56−/CD15− dual negative.

The results presented in the second part demonstrated the efficacy of the technique for transplanting myoblastic cells into infarcted heart tissues in the rat. It also makes it possible to determine the criteria required for good efficacy.

Finally, the third part reports clinical trials carried out in man of the transplantation of cells of muscular origin to reconstitute the myocardial tissues, to repair myocardial tissues, to generate metabolically active tissue, to generate tissue displaying a functional activity that was not present before this reconstitution. It also demonstrates that muscle cells can also play a role in the reshaping of heart tissue in man.

### EXPERIMENTAL SECTION

### CAPTIONS FOR THE FIGURES

FIG. 1: Graph of the LVEF values for the treated and control groups.

FIG. 2: Graph of the LVEDV values for the treated and control groups.

FIGS. 3A and 3B: Graph of the LVEF values after 1 month (3A) and LVEF values after 2 months, depending on risk categories.

FIG. 4: Linear regression showing the correlation between the functional improvement and the number of cells injected.

FIG. 5: Pre-operative (A) and post-operative (B) ultrasound scans: the systolic thickening of the initially akinetic posterior wall is clearly visible.

FIG. 6: Horizontal view of two ventricles in positron emission tomography (PET) imaging, showing the posterior wall of the left ventricle (transplanted zone at the bottom of the scan). Plates 6A and 6B (top) show homogeneous metabolic activity in the septal and anterior walls with a reduction in the metabolic activity in the posterior wall before the operation. Plates 6C and 6D (bottom) show the uptake of ₂fluoro¹⁸-deoxyglucose by the posterior wall after the operation.

FIG. 7: Stability of the characteristics of the cell populations (CD56+) depending on the successive expansions.

### A. METHOD FOR OBTAINING CELL POPULATIONS DERIVED FROM SKELETAL MUSCLE TISSUES

A.1. Materials, Solutions and Media Used

Medium A: Modified MCDB120 medium (23): L-valine replaced by D-valine, elimination of phenol red, elimination of thymidine.

Medium B: Medium A+20% irradiated fetal calf serum+antibiotics (100 IU/ml for penicillin and 100 μg/ml for streptomycin).

Medium C: medium B+bFGF (10 ng/ml)+dexamethasone (1 μM).

Solution D: PBS

Medium E: Medium A+bFGF (10 ng/ml)+dexamethasone (1 μM)+sterile human serum albumin (0.5%).

Solution F: Sterile isotonic saline solution, 0.9% NaCl.

Solution G: Solution F+4% human serum albumin and 7.5% DMSO (dimethylsulfoxide) final.

Solution H: Injection solution F+0.5% human serum albumin.

The method described below for obtaining cell populations involves 6 steps:
removal and mincing of the biopsy specimen
enzymatic dissociation and separation of individual cells by filtration
culture of the cells and cell expansion phases
identification of the cell types present at the different stages of culture by analysis of specific cell markers
harvesting of the cells
preparation and/or maintenance and/or survival and/or freezing the cells
preparation of the cell therapy product.

In the method, the cells are centrifuged at 160 g for 5 minutes. The cells are counted and the populations analysed using a Neubauer hemacytometer. In the expansion phases, the cells are incubated at 37° C. in an air-CO₂ incubator (95%-5%) with saturated humidity. The cells are observed using a phase-contrast, inverted microscope.

A.2. Results

A.2.1. Removal and Mincing of the Biopsy

The specimen is removed in the operating theater, in sterile medium, using an open system. A biopsy specimen of about 10-16 grams of skeletal muscle tissue is taken by the surgical team. The biopsy specimen is then cut into small cubes measuring 2 to 4 mm and then minced usign fine scissors in medium A. The minced tissue is then placed in a sterile bottle containing 25 ml of medium A.

The mincing step can also be carried out with assistance, using Medimachine® homogenizers with blades (distributed by Becton-Dickinson). In this system, fragments with a mass of less than 0.2 g are dissociated in sterile Medicon containers, after homogenization controlled by an electrical motor, for a duration of less than 5 minutes. Repeating the operation using several Medicon containers makes it possible to obtain a final yield of several grams of muscle. Table A, below, shows the proportions of CD56+, CD15+ and CDE34+ cell types obtained during the various stages of the culture of D0, D15, D20 and D26 (the proportion of CD34 cells being virtually nil at D15, this is not shown in the table for the steps after the beginning of culture on D0). Unexpectedly, it is found that the step of mincing the biopsy specimen is crucial for obtaining rapidly a population containing a dominant CD56+ myoblast type.

TABLE A
COMPARISON OF MINCING WITH
SCISSORS/MECHANICAL HOMOGENIZATION

| | | BIOPSY 1 | | BIOPSY 2 | |
|---|---|---|---|---|---|
| | | MINCED WITH | MECHANICAL | MINCED WITH | MECHANICAL |
| | | SCISSORS | HOMOGENIZAT [symbol] | SCISSORS | HOMOGENIZATI [symbol] |
| J0 | WEIGHT | 0.8 g | 0.8 g | 1.2 g | 1.4 g |
| | CNT 10 * 5 | 2.6 | 2.4 | 6.4 | 4.8 |
| | % CD34+ | 7.4 | 4.7 | 2.6 | 1 |
| | % CD56+ | 0.4 | 0 | 1.2 | 0.4 |
| | % CD15+ | 0 | 0 | 0.9 | 3.3 |
| PASSAGE 1 | DAY | D15 | D15 | D7 | D15 |
| | CNT 10 * 5 | 11.7 | 18.4 | 16.3 | 16.3 |
| | % CD56+ | 51.6 | 90.5 | 35.1 | 77.9 |
| | % CD15+ | 1.8 | 0.9 | 44.1 | 2.6 |
| PASSAGE 2 | DAY | D20 | D20 | D21 | D21 |
| | % CD56+ | 58 | 93.4 | 15 | 89.6 |
| | % CD15+ | 20.9 | 4.4 | 20 | 7.3 |
| PASSAGE 3 | DAY | D26 | D26 | D27 | D27 |
| | % CD56+ | 43.6 | 75.8 | 49.7 | 60.2 |
| | % CD15+ | 30.1 | 1.7 | 10.9 | 1.1 |

Different sample sizes have been tested for the performance of the method according to the invention, ranging from 0.13 g to 14.9 g. The results shown in the tables that follow show that the change in the proportions of the different types of population and the amplification of the number of cells change in a similar fashion for biopsy sizes of less than 1 g (Table B) to over 10 g (Table C).

TABLE B
Cultures from biopsies weighing more than 10 g

| | | D0 | | | | | | | |
|---|---|---|---|---|---|---|---|---|---|
| | PA- | BIOPSY | CNT | | | | | | |
| | TIENT | WEIGHT | (*) | % | % | % | % | % | % |
| NAME | AGE | (g) | 10 * 6 | CD56+ | CD15+ | CD34+ | CD10+ | CD45+ | HLADR+ |
| MYO 001 | 73 | 14.9 | 10 | 3.2 | 0.9 | 12.2 | 5.3 | 6.3 | ND |
| MYO 003 | 63 | 10.4 | 4.32 | 3.4 | 0.1 | 4.4 | ND | 0.1 | ND |
| MYO 004 | 67 | 13.9 | 10.25 | 32.4 | 1.5 | 9.2 | ND | 4 | ND |
| MYO 005 | 39 | 11.6 | 11.69 | 22.1 | 0.9 | 16 | ND | 3.9 | ND |
| MYO 006 | 55 | 12 | 16.4 | 28.7 | 0.16 | 8.9 | ND | 2.6 | 7.8 |

| | FIRST PASSAGE | | | | | | | |
|---|---|---|---|---|---|---|---|---|
| | | CNT (*) | % | % | % | % | % CL1 | % |
| NAME | DAY | 10 * 6 | CD56+ | CD15+ | CD34+ | CD10+ | HLA+ | DESMIN+ |
| MYO 001 | D8 | 19.46 | 48 | 49.8 | 3.4 | 76.5 | ND | ND |
| MYO 003 | D9 | 14.25 | 67.7 | 29 | 1.1 | 45.4 | 79 | ND |
| MYO 004 | D9 | 3.4 | 76.9 | 33 | 13 | 42 | 64 | 23.1 |
| MYO 005 | D7 | 31 | 71.5 | 28.5 | 11.2 | 8.1 | 91.7 | 80.8 |
| MYO 006 | D8 | 26.45 | 82 | 18.9 | 5 | 4.5 | 95.6 | ND |

SECOND PASSAGE

| | | CNT (*) | | | % | % CL1 |
|---|---|---|---|---|---|---|
| | DAY | 10 * 6 | % CD56+ | % CD15+ | DESMIN+ | HLA+ |
| MYO 001 | D13 | 315 | 58.3 | 34.1 | ND | 63 |
| MYO 003 | D13 | 156 | 87.1 | 11.3 | 87.5 | ND |
| MYO 004 | D15 | 115.2 | 97.5 | 5.4 | 88.3 | 95.5 |
| MYO 005 | D10 | 244.4 | 89.9 | 9.2 | 85.9 | 94.3 |
| MYO 006 | D13 | 483.6 | 91.3 | 10.5 | 82.4 | 96.5 |

FINAL YIELD

| | | CNT (*) | | | % | % CL1 |
|---|---|---|---|---|---|---|
| | DAY | 10 * 6 | % CD56+ | % CD15+ | DESMIN+ | HLA+ |
| MYO 001 | D16 | 890 | 67.3 | 31.6 | 70.5 | ND |
| MYO 003 | D18 | 921.7 | 91.3 | 6.2 | 64.6 | ND |
| MYO 004 | D20 | 656.9 | 97.1 | 15.5 | 58.2 | 94 |
| MYO 005 | D14 | 992.7 | 95.2 | 4.3 | 78.2 | 95.8 |
| MYO 006 | D16 | 1210 | 84.9 | 10.5 | 84.8 | 96.4 |

(*) CNT: cell count

TABLE C
Cultures from biopsies weighing less than 0.5 g

| | | | PAS- | PAS- | |
|---|---|---|---|---|---|
| | | | SAGE 1 | SAGE 2 | PASSAGE 3 |
| BIOPSY | TREATMENT D | D0 | D10 | D14 | D16 |
| 5007 | CNT 10 * 5 | 1.2 | 11.2 | 64 | 266 |
| 0.23 g | % CD34+ | 28.8 | ND | ND | 0 |
| | % CD56+ | 1.95 | 68.6 | 87.6 | 89.5 |
| | % CD15+ | ND | 30.9 | 17.3 | 28.8 |
| | % VIABILITY | 88.9 | 96 | 100 | 98.7 |
| BIOPSY | TREATMENT D | D0 | D7 | D10 | D15 |
| 5008 | CNT 10 * 5 | 2.04 | 7 | 21.6 | 586.6 |
| 0.23 g | % CD34+ | 34 | ND | ND | 0 |
| | % CD56+ | 6.1 | 47.1 | 66.3 | 92.9 |
| | % CD15+ | ND | 44.6 | 30.1 | 9.5 |
| | % VIABILITY | 85.1 | 92 | 88 | 97.9 |
| BIOPSY | TREATMENT D | D0 | D7 | D10 | D14 |
| 5011 | CNT 10 * 5 | 2.1 | 9.8 | 35 | 285 |
| 0.2 g | % CD34+ | 26.2 | ND | ND | ND |
| | % CD56+ | 3.5 | 24.1 | 29.9 | 38 |
| | % CD15+ | ND | 70.8 | 63.8 | 60.5 |
| | % VIABILITY | 85.5 | 98 | 98 | 99 |
| BIOPSY | TREATMENT D | D0 | D6 | D11 | D17 |
| 5054 | CNT 10 * 5 | 2.8 | 6.6 | 7.1 | 370 |
| 0.45 g | % CD34+ | 10.7 | ND | ND | ND |
| | % CD56+ | 25.7 | 62.3 | 69.1 | 84.6 |
| | % CD15+ | ND | 31 | 26.9 | 14.5 |
| | % VIABILITY | 88.7 | 100 | 96 | 87 |
| BIOPSY | TREATMENT D | D0 | D7 | D8 | D15 |
| 5058 | CNT 10 * 5 | 7.7 | 9.4 | 13.6 | 534 |
| 0.19 g | % CD34+ | 44.3 | 21.4 | ND | ND |
| | % CD56+ | 8.4 | 23.2 | 27.5 | 72.2 |
| | % CD15+ | ND | 63.2 | 63.6 | 18.9 |
| | % VIABILITY | 95 | 82.1 | 100 | 97 |
| BIOPSY | TREATMENT D | D0 | D6 | D13 | D15 |
| 5060 | CNT 10 * 5 | 5.2 | 9 | 18.8 | 663 |
| 0.33 g | % CD34+ | 48.7 | ND | ND | ND |
| | % CD56+ | 7.9 | 42.3 | 52.8 | 89.7 |
| | % CD15+ | ND | 46.8 | 48.6 | 11.9 |
| | % VIABILITY | 94.4 | 100 | 99 | 98 |

Biopsies have been taken from patients between 15 and 73 years of age. The results reported in Table D, below, show that the method is applicable regardless of the age of the patient from whom the biopsy is taken.

TABLE D
Preparation of cells of muscular origin from biopsies taken from patients of different ages

| | Day 0 | | | | First passage | | Second passage | | Third passage | |
|---|---|---|---|---|---|---|---|---|---|---|
| | | Patient's age | Number | | Number | | Number | | Number | |
| Name | Weight (g) | (years) | 10 * 5 | % CD56+ | 10 * 6 | % CD56+ | 10 * 6 | % CD56+ | 10 * 6 | % CD56+ |
| MYO1 | 14.9 | 73 | 100 | 3.2 | 19.5 | 48 | 315 | 58.3 | 890 | 67.3 |
| MYO3 | 10.4 | 63 | 43 | 3.4 | 14.2 | 67.7 | 156 | 87.1 | 922 | 91.3 |
| MYO4 | 13.9 | 67 | 102 | 32.3 | 3.4 | 76.9 | 115 | 97.5 | 657 | 97.1 |
| MYO5 | 11.6 | 39 | 117 | 22.1 | 31 | 71.5 | 244 | 89.9 | 993 | 95.2 |
| MYO6 | 12 | 55 | 164 | 28.7 | 26.5 | 82 | 483 | 91.3 | 1210 | 84.9 |
| 4929 | 0.19 | 15 | 1 | ND | 1.6 | 64.3 | 4.7 | 75.4 | 56 | 82.4 |
| 5008 | 0.24 | 45 | 2 | 6.1 | 0.7 | 47.1 | 2.2 | 66.3 | 59 | 92.9 |
| MOS | 3.6 | 84 | 110 | ND | 75 | 93.4 | 240 | 96.4 | 565 | 93.7 |
| CEL | 3.0 | 51 | 45 | ND | 42 | 51.8 | 120 | 63.7 | 548 | 68.7 |

The biopsies can be kept for 90 h at 4° C. or frozen in an equilibrated saline solution before being cultured. Tables E and F, below, show that the viability of the cells and the changing proportions of the various population types are not significantly affected after storing the biopsy for 90 h at 4° C. or after freezing.

TABLE E
Culture of a biopsy kept for 90 H at 4° C. in an equilibrated saline solution
and preparation of muscle cells according to the method.

| WEIGHT: 1.05 g | D0 | PASSAGE 1 (D7) |
|---|---|---|
| CNT 10 * 6 | 0.8 | 4.9 |
| % CD34+ | 7.7 | NS |
| % CD56+ | 6.5 | 58.7 |
| % CD15+ | 8.4 | 37.7 |
| % VIABILITY | 90.5 | 95 |

NS: not significant

TABLE F
Culture from a thawed biopsy

| | | | PAS- | PAS- | PAS- |
|---|---|---|---|---|---|
| | | | SAGE 1 | SAGE 2 | SAGE 3 |
| BIOPSY | TREATMENT | D0 | D18 | D20 | D25 |
| 4929 | CNT 10 * 5 | 0.97 | 16.2 | 47.3 | 55.7 |
| thawed | % CD34+ | 20.5 | ND | 0.04 | 0 |
| healthy | % CD56+ | 44.9 | 64.3 | 75.4 | 82.4 |
| muscle | % CD15+ | ND | 27.1 | 24.5 | 24.5 |
| WEIGHT: 0.19 g | % VIABILITY | 90.8 | 100 | 96.8 | 96.7 |

The culture method can be carried out using biopsies from healthy subjects or patients presenting with a disease. The results shown below in Table G show, in particular, that the method according to the invention can be used to prepare cells of muscle origin from a patient suffering from Duchenne's muscular dystrophy.

TABLE G
Culture from a thawed biopsy from a patient suffering
from Duchenne's muscular dystrophy.

| | | | PAS- | PAS- | PAS- | PAS- | PAS- |
|---|---|---|---|---|---|---|---|
| | | | SAGE 1 | SAGE 2 | SAGE 3 | SAGE 4 | SAGE 5 |
| BIOPSIE | STAGE | D0 | D7 | D14 | D21 | D26 | D29 |
| 4964 | CNT 10 * 5 | 1.58 | 1.78 | 11.6 | 473.6 | 2411.2 | 6397 |
| DE 24 H | % CD34+ | 53.9 | ND | 0.3 | 0 | ND | 0 |
| striated | % CD56+ | 5 | 24.6 | 78.1 | 73.7 | 60 | 52.5 |
| paravertebral muscle | % CD15+ | 0.4 | 30.4 | 12.7 | 15.5 | 26.4 | 36.1 |
| WEIGHT: 0.136 g | % VIABILITY | ND | 94 | 82.5 | 91.2 | 95.6 | 98 |

The results shown below show that the method can be carried out using all types of muscle biopsy. In particular, biopsies have been obtained from various muscles: the paravertebral, the anterior tibialis, the longus fibularis, the common extensor of the toes, the peroneus longus, the posterior tibialis and the soleus. The results obtained for the various biopsies are reported in Table H.

TABLE H
Preparation of cells of muscular origin from biopsies taken from
various anatomical locations

| | Day 0 | | | | | First passage | | | | |
|---|---|---|---|---|---|---|---|---|---|---|
| Name | Weight (g) | Source | Number 10 * 5 | % CD56+ | % CD34+ | Number 10 * 6 | % CD56+ | % CD34+ | % CD15+ | % Class I+ |
| MYO1 | 14.9 | vastus lateralis | 100 | 3.2 | 12.2 | 19.5 | 48 | 3.3 | 49.8 | ND |
| MYO3 | 10.4 | vastus lateralis | 43 | 3.4 | 4.35 | 14.2 | 67.7 | 1.1 | 29 | 79 |
| MYO4 | 13.9 | vastus lateralis | 102 | 32.3 | 9.2 | 3.4 | 76.9 | 13 | 33 | 64 |
| MYO5 | 11.6 | vastus lateralis | 117 | 22.1 | 16 | 31 | 71.5 | 11.2 | 28.5 | 91.7 |
| MYO6 | 12 | vastus lateralis | 164 | 28.7 | 8.85 | 26.5 | 82 | 5 | 18.9 | 95.6 |
| 4929 | 0.19 | paravertebral | 1 | NA | 20.5 | 1.6 | 64.3 | ND | 27.1 | ND |
| 5007 | 0.23 | anterior tibialis | 1.2 | 1.95 | 28.8 | 1.1 | 68.6 | ND | 30.9 | ND |
| 5008 | 0.24 | longus fibularis | 2 | 6.1 | 34 | 0.7 | 47.1 | ND | 44.6 | ND |
| 5011 | 0.2 | Common extensor | 2.1 | 3.5 | 26.2 | 1 | 24.1 | ND | 70.8 | ND |
| | | of the toes | | | | | | | | |
| 5054 | 0.45 | longus peroneus | 2.8 | 25.7 | 10.7 | 0.6 | 62.3 | ND | 31 | ND |
| 5058 | 0.19 | posterior tibialis | 7.7 | 8.4 | 44.3 | 0.9 | 23.2 | 21.4 | 63.2 | ND |
| 5060 I | 0.33 | soleus | 5.2 | 7.9 | 48.7 | 0.9 | 42.3 | ND | 46.8 | ND |

| | Second passage | | | | | Third passage | | | | |
|---|---|---|---|---|---|---|---|---|---|---|
| Name | Number 10 * 6 | % CD56+ | % CD15+ | % Class I+ | % Desmin+ | Number 10 * 6 | % CD56+ | % CD15+ | % Class I+ | % Desmin+ |
| MYO1 | 315 | 58.3 | 34.1 | 63 | ND | 890 | 67.3 | 31.5 | ND | 70.5 |
| MYO3 | 156 | 87.1 | 11.3 | ND | 87.5 | 922 | 91.3 | 6.2 | ND | 64.6 |
| MYO4 | 115 | 97.5 | 5.4 | 95.5 | −88.3 | 657 | 97.1 | 15.5, | 94 | 58.1 |
| MYO5 | 244 | 89.9 | 9.2 | 94.3 | 85.9 | 993 | 95.2 | 4.3 | 95.8 | 78.2 |
| MYO6 | 483 | 91.3 | 10.5 | 96.5 | 82.4 | 1210 | 84.9 | 10.5 | 96.4 | 84.8 |
| 4929 | 4.7 | 75.4 | 24.5 | ND | ND | 56 | 82.4 | 24.5 | ND | ND |
| 5007 | 6.4 | 87.6 | 17.3 | ND | ND | 27 | 89.5 | 28.8 | ND | ND |
| 5008 | 2.2 | 66.3 | 66.3 | ND | ND | 59 | 92.9 | 9.5 | ND | ND |
| 5011 | 3.5 | 29.9 | 63.8 | ND | ND | 28 | 38 | 60.5 | ND | ND |
| 5054 | 0.7 | 69.1 | 26.9 | ND | ND | 37 | 84.6 | 14.5 | ND | ND |
| 5058 | 1.4 | 27.5 | 63.6 | ND | ND | 53 | 72.2 | 18.9 | ND | ND |
| 5060 | 1.9 | 52.8 | 48.6 | ND | ND | 66 | 89.7 | 11.9 | ND | ND |

A.2.2. Enzymatic Dissociation and Separation of Individual Cells by Filtration

The flask containing the minced tissue was centrifuged at room temperature. The supernatant was removed by aspiration. The weight of the minced tissue was determined by weighing on a balance tared using an empty flask. The minced tissue was rinsed using 25 ml of medium A. After allowing the mince to settle, the supernatant was removed by aspiration.

A solution of Liberase (Roche-Boehringer) was prepared according to the Manufacturer's instructions, then repackaged and frozen at a concentration of 10 mg/ml. The Liberase was thawed when required and then added to the minced tissue at a concentration of 0.1 mg/ml, in a volume of 10 ml per gram of tissue. The bottle was placed in the oven at 37° C. for a period of 60 minutes. The bottle was shaken manually every 5 to 10 minutes (diffusion of the enzyme and gentle mechanical dissociation). The suspension was then centrifuged. The supernatant was removed by aspirating with a pipette. This first digestion product was then incubated in a 0.25% solution of trypsin. Ten ml of enzymatic solution was used per gram of initial tissue. The suspension was digested in the oven for 20 minutes at 37° C., shaking manually every 5 minutes. It was then aspirated and ejected using a 25 ml pipette. 10% irradiated fetal calf serum (Hyclone) was then added to neutralize the enzymatic activity.

The digestive product was then filtered through a 100-μm strainer, then a 40-μm strainer, under gravity, in order to separate the dissociated cells from the residual tissues. One strainer was used per gram of tissue (Falcon cell strainer). The filtrate was centrifuged at 300 g for 5 minutes. After discarding the supernatant, the pellets were washed with medium B, and then centrifuged.

The supernatant was removed by aspiration. The pellet was then resuspended in 10 l of medium C. A volume of 100 μl was taken for counting. An aliquot was set aside for estimating the viability by means of cytofluorimetry (propidium iodide).

A.2.3. Culturing the Cells and Culture Expansion Phases

After beign removed from the packaging, the cells were transferred into the culture system. The culture system consists of a culture tray (Nunc single tray) with an area of 600 cm². The tray was filled through an opening provided for the purpose and stoppered with a sterile, disposable stopper. The cultures were incubated at 37° C. in a controlled air-CO₂ (95%-5%) atmosphere saturated with humidity.

The day after the beginning of culture, the tray was drained for the first time to remove dead cells and muscle debris. An empty bag was connected to one of the two openings in the culture system. The medium was removed by draining under gravity and replaced by 120 ml of medium C, which was added to the culture. Medium C was replaced after 120 to 192 hours. The decision to carry out expansion was taken when the cells reach 20 to 50% confluence or when the first myotubes appear (about 8 days after starting the culture).

After draining the medium, the cells were washed with 50 ml of solution D by gentle manual stirring. Solution D was drained off and then 20 ml of irradiated trypsin solution (0.25%) was added. The bottle was incubated for 5 minutes at 37° C. The cells were harvested in a 40 ml bag. The action of the trypsin was neutralized by adding 10% fetal calf serum. The serum was injected into the bag using a syringe. The cells were centrifuged. The supernatant was removed by transferring into another draining bag, to which it was connected by a sterile link. The pellet was suspended in 30 ml of medium C to wash the cells, and the cells were then centrifuged. The supernatant was removed by transferring into another draining bag, connected by a sterile link. The cell pellet was resuspended in 20 ml of medium C. An aliquot was taken for counting and analyzing the populations. The viability was estimated using a cytofluorimeter. The cells were then transferred into a bag containing 500 or 756 ml of medium C, then reseeded into two or three double-tray units (Nunc double tray) with a total surface area of 1200 or 1800 cm². The cells are then put to incubate. The decision to carry out a third expansion was taken when the degree of confluence of the cells reached 60 or 70%, or when the first myotubes appeared. For this series of expansions, 10-tray dishes (Nunc multi-tray) were used. After draining off the medium, the cells were washed using 100 ml of solution D. After draining the washing solution, the cell layer was detached and the enzymatic dissociation of the cells were carried out by adding 50 ml of irradiated trypsin (0.25%) to each tray. The preparations were incubated for 5 minutes at 37° C. The cells were harvested in sterile 300 to 600-ml bags. The action of trypsin is neutralized by adding 10% volume/volume fetal calf serum. The cells were centrifuged and the supernatant was removed by connecting to a draining bag.

The cell pellet was resuspended in 50 ml of medium C and then transferred into one or two bottles containing 1200 or 2400 ml of medium C by a sterile link. One or two 10-tray culture dishes (Nunc multitray) were seeded with 1200 or 2400 ml of the cell preparation. It is impossible to monitor growth visually in multitray dishes, and so a single-tray culture dish (Nunc single-tray) was also seeded with 110 ml of the cell preparation. This tray made it possible to monitor the culture and the degree of confluence of the cells visually on a daily basis. The cells are then incubated. The culture was continued for 3 to 5 days. The day before the cultures were harvested, the medium was removed by draining into a sterile bag and replaced by an equal volume of medium E. The decision to carry out the final harvest was taken when the degree of confluence of the cells reached 90% or when the first myotubes appeared.

A.2.4. Identification of the Cell Types Present at Different Stages of the Culture by Analyzing Specific Cell Markers

The characterization was based on cytofluorimometric flow analysis (FACS). Antibodies against the following human cell surface antigens were used: CD5, CD10, CD11, CD13, CD14, CD15, CD16, CD18, CD19, CD20, CD28, CD31, CD34, CD38, CD40, CD40-ligand, CD44, CD45, CD56, CD62, CD71, CD80, CD86, CD90, CD105, CD117, CD138. Antibodies directed against the following antigenic structures have also been used: CD138, Class-I HLA, HLA-DR, ELAM, ICAM, LECAM, Stro-1, S-endo-1; VCAM, VLA2, VLA3, VLA4, VLA5, VLA6.

The analysis of the expression of desmin, an intracellular protein, was carried out as follows:

After suspending in PBS, the cells were fixed and permeabilized by adding 10 volumes of methanol at 4° C. for 5 minutes and then centrifuged. After washing off any residual methanol and centrifuging, the cells were resuspended in PBS containing the antibody raised against desmin (Dako, clone D33, 1/100) and incubated for 15 minutes. After washing and centrifuging, the cells were incubated for 15 minutes in the presence of the secondary antibody against the primary antibody, coupled to a fluorophore. After washing and centrifuging, the cells were analysed by FACS. Table I below lists the characteristics of the main markers used and the corresponding cell types (cell types).

TABLE I
Presentation of some of the cell markers used in the method and the
cell types that express them.
CONVENTIONAL CHARACTERISTICS OF THE MARKERS USED

| MARKER | CELL TYPE |
|---|---|
| CD10 | pre-B lymphocytes, neutrophils |
| CD13 | monocytes, myeloid cells |
| CD15 | monocytes, macrophages, granulocytes, eosinophils |
| CD16 | NK, sub-pop. of T-lymphocytes, neutrophils |
| CD34 | progenitors |
| CD38 | LT activities, stem cell, sub-pop. L, T, B, NK |
| CD40 | activated CD4+ T-lymphocytes |
| CD44 | Anti-HCAM |
| CD45 | leukocytes |
| CD56 | NK, sub-pop T-lymphocytes |
| CD71 | proliferating cells |
| CD117 | progenitors |
| HLA CL1 | class-1 MHC antigen |
| HLA CL2 | class-2 MHC antigen |
| VLA3 | B-lymphocytes |
| VLA5 | memory T-lymphocytes, monocytes, platelets |
| VLA6 | thymocytes, memory T-lymphocytes; monocytes |
| DESMIN | muscle cells |

The cell populations were analysed at various stages during the culture process:

On day D0 corresponding to the beginning of the culture of the freshly prepared population.

Several series have been prepared in unit bottles cultured in parallel with the single tray unit on D0, so as to be able to monitor the progress of the cultures daily by sampling one or more bottles per day.

On day 1 (D1), the non-adherent fraction (supernatant) and the adherent fraction (obtained by trypsination) were analysed separately. It is of interest to note that these two fractions were found to contain populations with differing characteristics.

From D2 to D9, the adherent cells were analysed every day in order to monitor the change in cell types over time.

In parallel, the cells obtained by the mass production were analysed at each expansion step and then at the time of the final harvest. The results show that at equivalent dates of harvesting, the characteristics obtained in the trays and independent bottles were the same.

The data obtained from identifying the cell types during the differentiation kinetic analysis are shown in tables JA and JB below and their essential characteristics are described below.

TABLE JA
Identification of cell types over time (as a percentage of the
total cell population) during culture stages D0 to D3.
Differentiation kinetics (percentages)

| MARKERS | D0 | D1 | SN D1 | D2 | D3 |
|---|---|---|---|---|---|
| CD34+ | 38.5 (25-75) | 77 (69-79) | 39 (25-67) | 74 (64-75) | 74 (61-75) |
| CD34− | 61.5 (25-75) | 23 (20-30) | 61 (33-75) | 26 (25-30) | 26 (25-49) |
| CD34+CD10+ | 14 (10-18) | 27 (25-29) | 2.5 (2-3) | 24 (20-40) | 11 (7-17) |
| CD34+CD10− | 25 (23-31) | 47 (40-54) | 30.5 (24-37) | 39 (36-54) | 59 (44-63) |
| CD34+CD45+ | <5 | ND | 0.5 (0-1) | ND | ND |
| CD34+CD56+ | 0 | 0 | 0 | 0.3 (0.3-0.3) | 0.7 (0.4-2) |
| CD34+DR+ | 14 | ND | 21.5 (16-27) | ND | ND |
| CD34+DR− | 28 | ND | 12 (12-12) | ND | ND |
| CD34+CD15+ | ND | ND | ND | ND | ND |
| CD56+ | 4.7 (0-26) | 4.4 (0.3-15) | 1 (0-5) | 11 (7.7-20) | 14 (12-35) |
| CD15+ | 1.5 (0-6) | 3 (1.5-6) | 3.6 (0.1-12) | 20.5 (20-21) | 25 (24-33) |
| CD56+CD15+ | 0.02 (0-0.3) | 0 | 0 | 4 (4-4) | 10 (6-14) |
| CD13+ | ND | ND | ND | ND | ND |
| CD44+ | 0 | ND | ND | ND | ND |
| CD117+ | 1.7 (0.16-1.7) | ND | 1.7 (0.2-2) | ND | ND |

TABLE JB
Identification of cell types over time (as a percentage of the total cell
population) during culture stages D4 to D8.

| MARKERS | D4 | D5 | D6 | D7 | D8 |
|---|---|---|---|---|---|
| CD34+ | 56 (36-67) | 35 (26-50) | 23 (13-47) | 2.7 (0.6-10) | 1.7 (0.4-3.4) |
| CD34− | 44 (33-63) | 65 (50-73) | 76 (53-87) | 97 (90-99) | 98 (96-100) |
| CD34+CD10+ | 9 (8-22) | 16.5 (3-34) | 16.8 (0.6-33) | 1.7 (0.2-2.7) | 0.4 (0.2-7.6) |
| CD34+CD10− | 35 (27-59) | 25 (15-35) | 14 (14-14) | 5.1 (0.4-9) | 1.3 (0.2-7.6) |
| CD34+CD45+ | ND | ND | ND | ND | ND |
| CD34+CD56+ | 3 (1.5-6) | 7.5 (1.5-12) | 3 (0.1-12) | 0.35 (0.2-0.6) | 0.4 (0.4-2.1) |
| CD34+DR+ | ND | ND | ND | ND- | ND |
| CD34+DR− | ND | ND | ND | ND | ND |
| CD34+CD15+ | ND | ND | ND | ND | ND |
| CD56+ | 34 (28-55) | 61 (50-68) | 73 (57-74) | 64 (52-87) | 68.4 (50.3-91) |
| CD15+ | 48 (46-48) | 48 (47-63) | 51 (29-64) | 36 (20-55) | 29 (12-54) |
| CD56+CD15+ | 19 (17-21) | 13 (9-25) | 10 (5-13) | 3.6 (2.6-6) | 1.4 (1-3) |
| CD13+ | ND | ND | 96 (76-97) | 99 (94-99) | 99 (96-99) |
| CD44+ | ND | ND | ND | ND | ND |
| CD117+ | ND | ND | ND | ND | ND |

A.2.4.1. Characteristics of the Cell Preparations at D0

The method yields 3×10⁵ to 4×10⁶ cells per gram of tissue on D1. The majority cell types were CD34+. The CD34+ cell types consist of 14% CD34+/CD10+, 25% CD34+/CD10−, 0% CD34+/CD56+, 14% CD34+/DR+ and 28% CD34+/DR−. Most of the CD34+ populations were CD45−. The minority populations express CD44, CD45, CD56, CD117, Class-I HLA. Suprisingly, the characterization of a large number of progenitor cells (in absolute terms and relative to the population) makes it possible to envisage harvesting cells at this stage so that they can be used as a cell therapy product for reconstituting many tissues, not only muscle tissues, but also hematopoietic, bone, adipose, cartilaginous or vascular tissues.

A.2.4.2. Characteristics of the Adherent Cell Preparations on D1

Most of the cells were CD34+. The population consisted of CD34+/CD10+ (27%) and CD34+/CD10− (47%). CD13 appears. The preparation was negative for CD117 and CD45. The CD15+ and CD56+ cell types constituted minority types.

A.2.4.3. Characteristics of the Cell Preparations Present in the Supernatant on D1

A high proportion of the cells were CD34+. The population consisted essentially of CD34+/CD10−. A CD117+ population (<5%) was present and expressed CD45+. Some minority populations were present: CD38+ (15%), CD45+, few CD15+ or CD56+ and Class-II HLA+.

A.2.4.4. Characteristics of the Change in Markers During Culture

The change was characterized by a progressive increase in the proportion of CD15+ and/or CD56+ cells and a fall in CD34+ cells. Over time, a progressive shift from a CD34+ population to a CD15+ population can be observed. The proportion of the CD13+, CD44+ and CD71+ populations increased over time. Minority CD138+, Class-II HLA+ and CD38+ populations were observed.

At the end of the expansion process, three populations predominated: CD56+, CD15+ and CD56−/CD15−. The CD56+ population expressed CD10, CD13, CD44, desmin and Class-I HLA. These are specific myoblastic cell markers. The CD15+ population expressed CD13, Class I and partially CD10. In the CD56−/CD15− population, one fraction expressed desmin and the other did not. Some markers were expressed more weakly and in a variable fashion; CD71, VLA3, VLA5, VLA6, CD16+ and CD40L. The CD34+, CD38+, CD45+ and Class-II HLA+ populations have disappeared or constitute a tiny minority.

A.2.4.5. Characteristics of the Cells After Depleting the CD34+ Fraction

Table K below shows the characteristics of the cells obtained after the first passage, by comparing various starting conditions. Eight independent experiments are shown. After depleting the CD34+ fraction, the method makes it possible rapidly to produce a strongly predominant cell population consisting of cells expressing CD56. In particular, the proportion of cells expressing CD56 is greater than can be obtained from an undepleted biopsy.

TABLE K
Depletion or enrichment with the CD34+ cells present in the
muscle biopsy specimen

| | % CD56+ during first passage | | | | | | | |
|---|---|---|---|---|---|---|---|---|
| | Exper- | Exper- | Exper- | Exper- | Exper- | Exper- | Exper- | Exper- |
| | iment 1 | iment 2 | iment 3 | iment 4 | iment 5 | iment 6 | iment 7 | iment 8 |
| Unseparated | ND | 72% | 87% | 41% | 48% | 67% | 70% | 38% |
| fraction | | | | | | | | |
| CD34− | 93% | 98% | 97% | 82% | 93% | 93% | 86% | 57% |
| depleted | | | | | | | | |
| fraction | | | | | | | | |
| CD34− | 61% | 36% | 37% | 1% | 8% | 28% | 4% | 12% |
| enriched | | | | | | | | |
| fraction | | | | | | | | |

A.2.5. Harvesting the Cells

The following protocol describes the harvesting of the cells during the final stage to obtain a population containing a majority of myoblasts. However, the protocol allows the specialist to use it at to harvest cells any differentiation stage chosen, depending on which cell population is sought.

After draining off the medium, the cells were washed using 500 ml of solution D (for the multitray units), 50 ml for the single unit or 100 ml for the double units. The washing solution was drained off and 200 ml of irradiated trypsin (0.25%) added to the multitray units (20 ml to the single unit, 40 ml to the double units). The preparation was incubated for 5 minutes at 37° C. The cells were harvested in a 500-ml bag. The action of trypsin was neutralized by adding 10% fetal calf serum injected using a syringe. The cells were washed as follows: the cells were centrifuged. The supernatant was removed and the cells resuspended in 300 ml of solution F and then centrifuged. The supernatants were discarded. Two further washing steps of the type just described, were carried out. The purpose of this repeated washing was to remove the trypsin, any animal proteins still present and the recombinant bFGF. During the third washing process, an aliquot was set aside for counting the cells, estimating their viability and quality and for microbiological quality controls. The cells can be concentrated in solution H in order to obtain a suitable suspension for the intended clinical use.

After centrifuging, the cells were resuspended in an isotonic solution at a concentration of 1.5×10⁸ cells/ml. Finally, they were aspirated into a 10-ml syringe. The cells were taken for injection in sterile syringes. The type of needle used for the injection depends on the target tissue. For direct intramyocardial injection, a 25 to 30-gauge needle with a right-angled bend was used specifically.

A.2.6. Production Yields and Characteristics of the Cell Types

Table L below summarizes the results obtained during the implementation of the method according to the invention from various biopsies taken from three different patients.

The cells were initially produced in single, double or multi-tray units up to the third expansion inclusive. The expansions were then carried out by dividing the populations and reseeding into 25 cm² culture dishes. At each passage, most of the cells were used for characterizing and counting, and a known number of cells used for seeding and expanding. The number of cumulative expansions can be calculated to make it possible to obtain about 100 billion cells between the eighth and ninth expansion.

Table L shows the yields in terms of the number of cells obtained and in terms of the proportion of CD56+ or desmin+ type cells in the population at the various stages of expansion for the three patients, designated as MYO 003, MYO 004 and MYO 005 respectively.

TABLE L
YIELD OF THE PROCESS IN TERMS OF THE NUMBER OF EXPANSIONS

| | | PASSAGE | | | | | | | |
|---|---|---|---|---|---|---|---|---|---|
| | D0 | 1 | 2 | 3 | 4 | 5 | 6 | 7 | 8 |
| MYO003 | | | | | | | | | |
| % CD56+ | 3.4 | 67.7 | 87.1 | 91.3 | 87 | 89.8 | 89.7 | 71.1 | 76.5 |
| % DESMIN+ | ND | ND | 87.5 | 64.6 | 86.2 | 88.2 | 88.5 | 66 | 64 |
| CNT CULTURED (10*6) | 4.32 | 4.32 | 14.25 | 156 | 3.4 | 0.3 | 1.2 | 0.59 | 0.35 |
| CNT OBTAINED (10*6) | * | 14.25 | 156 | 921 | 5.9 | 3.96 | 1.78 | 1.06 | 0.35 |
| PROLIFERATION | * | ×3.3 | ×10.9 | ×5.9 | ×1.73 | ×13.2 | ×1.48 | ×1.79 | ×1 |

THEORETICAL NUMBER OF CELLS AFTER 8 PASSAGES 55.46 × 10*9

| | | PASSAGE | | | | | | | |
|---|---|---|---|---|---|---|---|---|---|
| | D0 | 1 | 2 | 3 | 4 | 5 | 6 | 7 | 8 |
| MYO004 | | | | | | | | | |
| % CD56+ | 32.4 | 76.9 | 97.5 | 97.1 | 96 | 88.7 | 72.2 | 75 | 85 |
| % DESMIN+ | ND | 23.1 | 88.3 | 58.2 | 88.1 | 68 | ND | 79.4 | 70.4 |
| CNT CULTURED (10*6) | 10.25 | * | 3.4 | 115.2 | 0.18 | 0.5 | 0.22 | 0.11 | 0.32 |
| CNT OBTAINED (10*6) | * | 3.4 | 115.2 | 656.9 | 1.58 | 0.66 | 0.34 | 0.96 | 1.14 |
| PROLIFERATION | * | * | ×33.9 | ×5.7 | ×8.78 | ×1.32 | ×1.55 | ×8.73 | ×3.56 |

THEORETICAL NUMBER OF CELLS AFTER 8 PASSAGES 366.8 × 10*9

| | | PASSAGE | | | | | | | |
|---|---|---|---|---|---|---|---|---|---|
| | D0 | 1 | 2 | 3 | 4. | 5 | 6 | 7 | 8 |
| MYO005 | | | | | | | | | |
| % CD56+ | 22.1 | 71.5 | 89.9 | 95.2 | 96.4 | 89.1 | 91.5 | 68.8 | 82.7 |
| % DESMINE+ | ND | 80.8 | 85.9 | 78.2 | 74.1 | 89 | 89.6 | 88.7 | 83.2 |
| CNT CULTURED (10*6) | 11.69 | 11.69 | 31 | 244.4 | 0.2 | 0.45 | 0.46 | 0.4 | 0.53 |
| CNT OBTAINED (10*6) | * | 31 | 244.4 | 993 | 1.36 | 1.7 | 1.84 | 1.6 | 1.13 |
| PROLIFERATION | * | ×2.65 | ×7.88 | ×4.06 | ×6.8 | ×3.8 | ×4 | ×3.5 | ×2.13 |

THEORETICAL NUMBER OF CELLS AFTER 8 PASSAGES 763.7 × 10*9

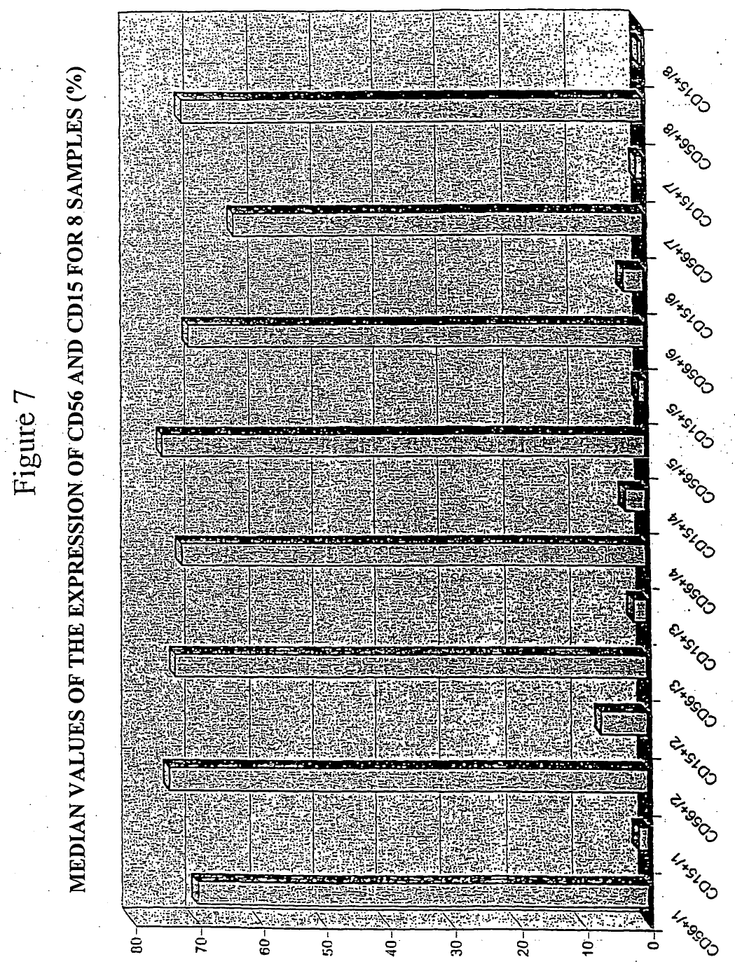

The histogram in FIG. 7 shows the median values of the expression of CD56 and CD15 in 8 samples during the various expansion phases.

The results shown in FIG. 7 show that the proportions of the CD56+ and CD15+ cell types obtained were relatively similar in the various biopsies, and did not change during the various expansions. In particular, it can be seen that the CD56+ cells remain dominant throughout the expansion phases.

These data also show that after identifying the optimal stages for harvesting for the various target cell types, the specialist can reproduce the method according to the invention without the cell characterization step described in the method according to the invention and increasing the number of expansion phases so as to obtain a large number of cells containing a specific predominant cell type, notably type CD56+ cells.

A.2.7. Freezing the Cell Therapy Product

In order to make it possible to use the cells thus prepared over a period of time, it may be advantageous to freeze them under conditions such that when they were subsequently thawed, a sufficient proportion of the cells survives, preferably over 90%.

By way of example, the cells were suspended in the freezing medium (solution G) and transferred into two sterile freezing bags, at a concentration of between 10⁷ and 2×10⁷ cells/ml or in cryofreezing tubes at a concentration of between 1×10⁶ and 5×10⁶/ml. The cells were frozen using a device (Digicool or Nicool), which produces a gradual and controlled lowering of temperature. The cells were stored in liquid nitrogen until they were thawed.

The cells were thawed in a water bath at 37° C. The cell preparations were washed twice, using an isotonic saline solution. The rinses were carried out via a sterile link to bags containing the isotonic solution and to the draining bags. An aliquot was set aside for estimating the cell viability and quality.

### B. FACTORS AFFECTING THE FUNCTIONAL PROPERTIES OF THE TRANSPLANTATION OF CELLS OF AUTOLOGOUS MUSCULAR ORIGIN FOR THE TREATMENT OF MODELS OF MYOCARDIAL ISCHEMIA

B.1. Materials and Methods

B.1.1. Model of Myocardial Ischemia

Male Wistar rats, weighing 280 g were anesthetized with ketamine (50 mg/kg) and xylasine (10 mg/kg) and ventilated via the trachea. A thoracotomy was carried out. Infarction of the myocardium was obtained by ligature of the left coronary, using a 7/10 polypropylene thread.

B.1.2. Functional Tests

One week after the myocardial infarction, and one or two months after transplanting, left ventricular function was investigated using 2D-ultrasound.

Under ketamine (50 mg/kg) or xylasine (10 mg/kg) general anesthesia, the 2D (and M-mode) measurements were carried out using a commercial 15 MHz apparatus “linear array transducer” (Sequoia, Acuson Corp., Mountain View, Calif., USA) with a top frequency of 160 Hz. Longitudinal parasternal images were obtained so that the mitral and aortic valves and the apex can be clearly seen and therefore recorded.

Measurements of the length (L) of the major axis of the left ventricle and plots of the endocardiac zones (a) were made. The volume of the left ventricle at the end of diastole (LVEDV) and the volume of the left ventricle at the end of systole (LVESV) were calculated using the following equation: V=8×A²/(C×π×L). The ejection fractions of the left ventricle (LVEF) were then calculated: LVEF=(LVEDV−LVESV)/LVEDV). All measurements were taken from at least three beats and by two investigators processing the different groups while blinded.

B.1.3. Cell Culture

During the myocardial infarction process, the right and left anterior tibialis muscles were dissected so as to separate the tendon and the aponeurotic tissue from the muscle tissue. They were then minced, weighed and subjected to enzyme dissociation, using collagenase IA (2 mg/ml, Sigma Chemical Co., St. Louis, Mo., USA) for an hour and trypsin-EDTA (0.25%, GIBCO BRL, Gaithersburg, Md., USA) for 20 minutes.

The cells were harvested by sedimentation (7 min at 1200 rpm) and the enzymatic reaction was neutralized by adding 10% fetal calf serum. After passing over a 100-μm strainer and centrifuging, the supernatant was discarded and the cells resuspended in a medium consisting of F12 (HAM) with 20% fetal calf serum, 1% (v/v) penicillin-streptomycin (10,0000 IU/ml-10,000 μg/ml, GIBCO BRL) and 5 ng/ml bFGF (Sigma).

The initial seeding was carried out in 75 cm² culture flasks, and the cells incubated in air containing 5% CO₂ and saturated humidity.

The day they were transplanted, after culturing for 7 days, and after functional assessment of the ventricular ejection fractions using ultrasound, the cells were harvested by trypsination, washed and the viability tested. A sample was seeded into 12-well dishes in 2.0 ml of culture medium for counting. The cells were then washed in the injection medium (culture medium+0.5% BSA, Fraction V) and kept in ice until being transplanted. The cells were centrifuged, resuspended in 150 μl of injection medium and administered by sub-epicardial route into the infarcted zone.

B.1.4. Transplanting the Cells into the Infarcted Zone

Forty-four rats were included in this study and were divided into two groups: a control group and a treated group.

All the rats underwent surgery again one week after the myocardial infarction, under general anesthesia and with tracheal ventilation. All the rats were given 150 μl of the injection medium administered into the infarcted zone using a 30-gauge needle In the control group (n=23), the rats were given the injection medium alone. The treated group (n=21) received the suspension of cultured myogenic cells.

In each group, four risk categories were investigated with regard to the baseline ejection fraction LVEF: <25% (n=15), 25-35% (n=15) and >40% (n=16). This stratification makes it possible to obtain similar numbers of animals in each subgroup, making the statistical analysis is more accurate.

B.1.5. Immuno- and Histochemical Studies

One day after the transplantation, the cells seeded in the 12-well dish were fixed with methanol and cooled to −20° C. for 5 minutes. The non-specific marking was neutralized using a mixture of 5% horse serum (HS) and 5% fetal calf serum in PBS for 20 minutes. The cells were incubated with a mouse antibody to desmin (1/200 DAKO, A/S Denmark) for one hour and then with an anti-mouse antibody conjugated with the Cy3 marker (1/200, Jackson Immuno Research Laboratories, Inc) for one hour in darkness.

The cells were observed using an inverted microscope with phase contrast and fluorescent illumination. Several images were taken randomly. The proportion of myoblasts was calculated by dividing the number of desmin-positive cells by the total number of cells examined.

Three months after the last ultrasound scan (i.e. 2 months after transplantation), the rats were sacrificed by an overdose of ketamine and xylasine. The ventricles were isolated and cut in two along their longitudinal axis. Both parts were placed in isopentane and frozen in nitrogen. Thin 8-μm sections were prepared using a cryostat and the usual histological examinations carried out after staining with hematoxylin and eosin.

B.1.6. Statistical Analysis

All the data are expressed as the mean±SEM. All the analyses were carried out using appropriate software (Statview 5.0, SAS Institute Inc. Cary, N.C., USA). The critical threshold α for the analyses was set as p<0.05.

The comparisons of the continuous variables in the control and treated group and for each risk category were carried out using analysis of variance (ANOVA method), followed by a post-hoc test (Shceffe). Longitudinal studies comparing the ultrasound findings for each group before and one or two months after the intramyocardial injection were carried out using the paired test.

To test the relationships between the number of cells injected and heart function after transplantation, two variables were constructed: (LVEF after 1 month/LVEF) and (LVEF after 2 months/LVEF). The link was studied by calculating the F-ratio for the ANOVA regression and the R² coefficient adjusted for analyses of the linear regression.

Furthermore, the variability of the ultrasound tests within each group was observed from two series of measurements carried out on 10 rats chosen at random, using a Bland and Altman analysis.

B.2. Results

B.2.1. Characterization of the Suspension Injected

Of the 10,000 cells counted on the day of the transplantation, 50% positively expressed desmin. The number of cells injected was 3,500,000±500,000, ranging from 700,000 to 6.5×10⁶.

B.2.2 Functional Test after Transplanting Cells of Muscular Origin

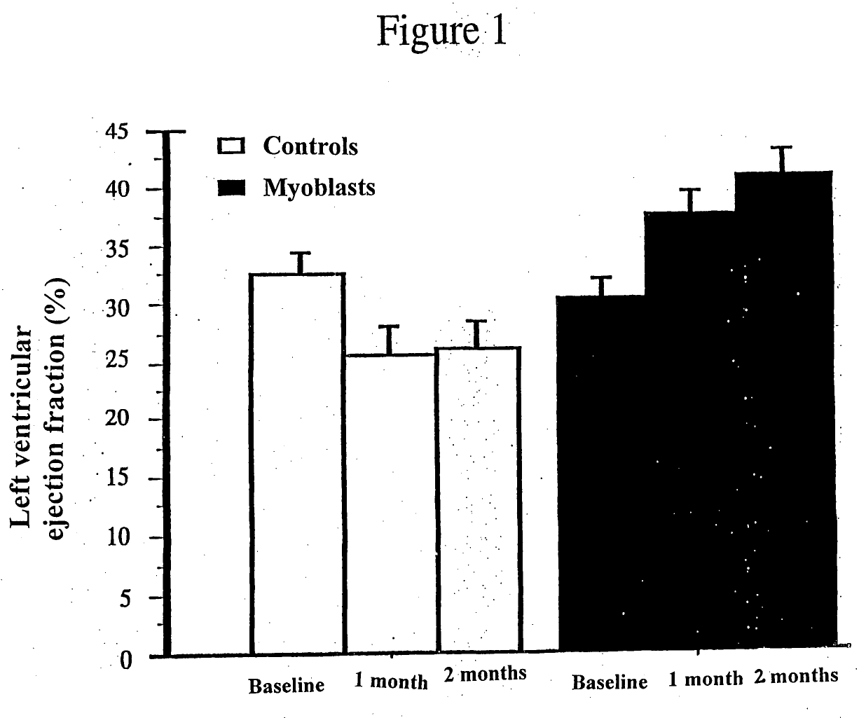

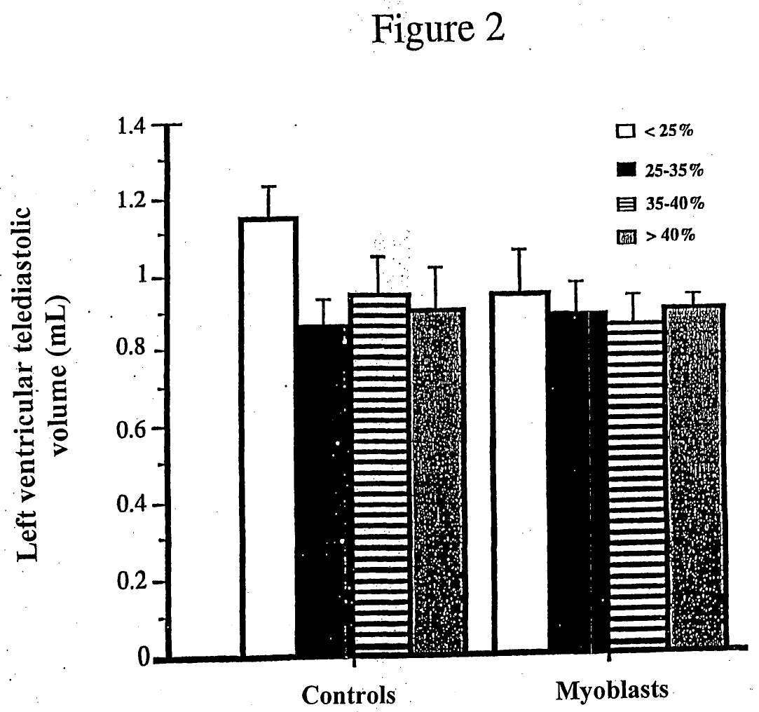

The ultrasound parameters at baseline were not significantly different in the different groups. On the contrary, most of the major differences between the groups were observed after transplantation. Thus, in the treated group, heart function was improved, as can be seen by comparing the LVEF with that of the control groups (FIG. 1). One month after the myocardial injection, significant differences could be seen between the two groups (37.52±1.92% versus 25.49±2.47%, p=0.0005). This finding was confirmed 2 months after the injection after the myocardial injection, significant differences could be seen between the two groups (40.92±2.17% versus 25.83±2.39%, p=0.0001). This improvement in LVESV in the treated group was essentially related to a smaller increase in LVESV compared to the ventricular dilatation, corresponding to the variable LVEDV, which showed a similar increase in the two groups (FIG. 2).

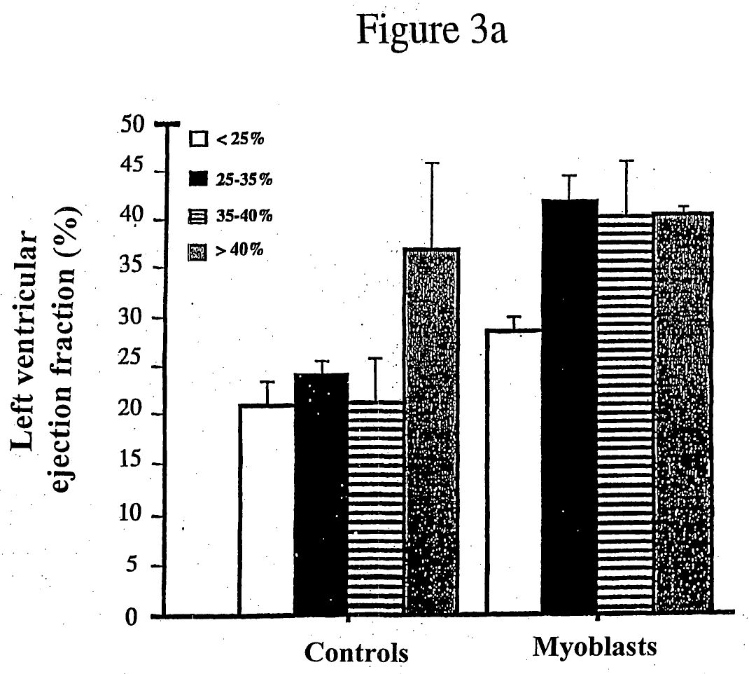

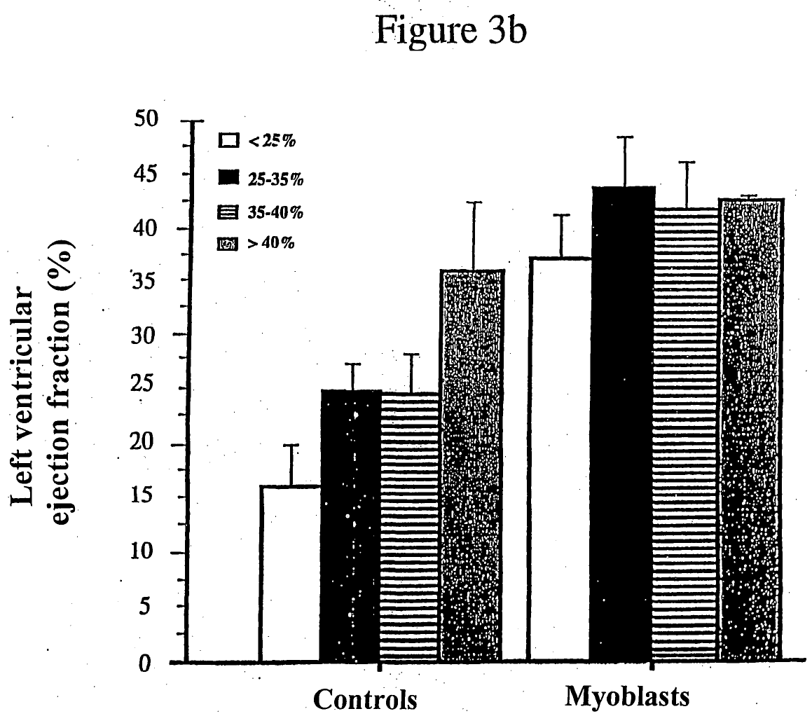

The longitudinal analyses of the two groups showed a substantial improvement of left ventricular function in the treated group (FIG. 3). Significant differences were observed by comparing LVEF after 1 month and LVEF after 2 months to the baseline LVEF variable. Significant differences were also found by comparing LVEF after one month with LVEF after 2 months. Whereas LVEDV and LVESV had both increased relative to baseline after one month (p<0.0001 and p=0.0003 respectively) and after 2 months (p<0.001 and p=0.029 respectively), a stabilization and a reduction were found when the values after 2 months were compared with those after 1 month (p=0.78 and p=0.12 respectively). In the control group, there was a considerable reduction in LVEF with a significant increase in LVEDV was already visible after one month (p=0.0066 and p<0.001 versus baseline respectively). Both parameters gave similar values after 2 months.

When heart function (LVEF) was analysed by risk categories in terms of baseline LVEF, differences were found between the control and treated groups after one month in the two intermediate sub-groups (25-35% and 35-40%). This improvement in LVEF was confirmed in both subgroups 2 months after injection, but interestingly a beneficial effect was also found by comparing the treated group with the control group in the <25% risk group.

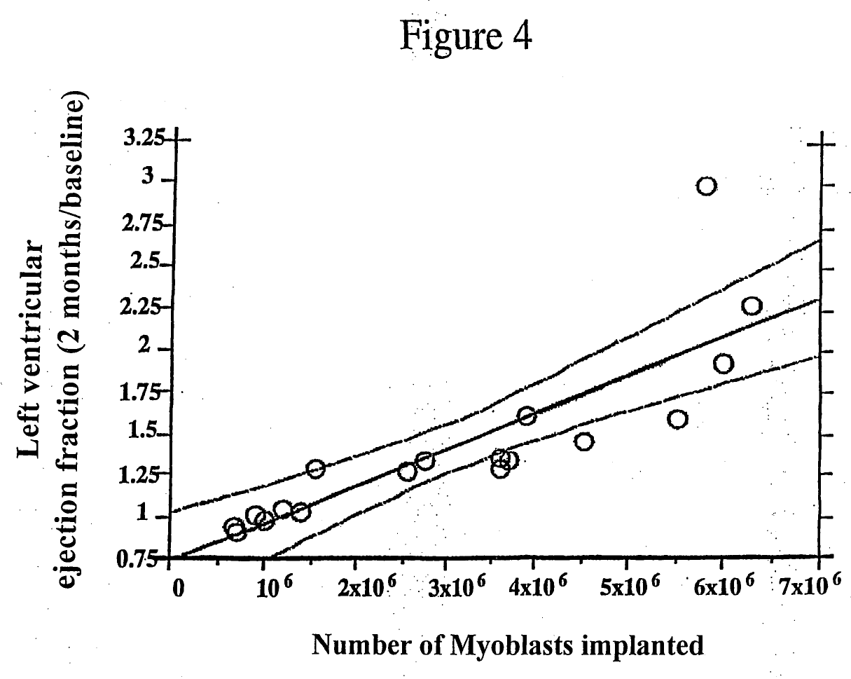

Finally, regression analysis revealed a significant link between the number of myoblasts transplanted and the LVEF ratios after one month (R²=0.675, p<0.0001) and after two months (R²=0.714, p<0.0001) (FIG. 4). When the data were analysed in terms of risk group, the impact after 2 months of the number of cells injected was also significant in the <25%, 25-35% and 35-45% subgroups (R²=0.836, p=0.0106, R²=0.928, p=0.0083 and R²=0.985, p=0.0076, respectively).

B.2.3. Cumulative Effects of Transplanting Cells of Muscular Origin and Treatment with an Angiotensin-Converting Enzyme Inhibitors (ACEI)

Currently, heart failure is managed by administering ACE inhibitors. It was therefore of interest to find out whether there is any synergism between transplanting cells of muscular origin obtained by the method according to the invention and the protective effect produced by ACE inhibitors.

Myocardial infarction was produced in 39 rats by ligature of the coronary arteries. Treatment with 1 mg/kg perindoprilat per day (an ACE inhibitor) was introduced immediately after the infarction, and continued without interruption until the animal was sacrificed. One week after the infarction, the animals underwent surgery again and were selected randomly to receive a sub-epicardial injection of 150 μl of culture medium alone (control group, n=21) or an equal volume containing cells of muscular origin, i.e. about 3×10⁶ cells cultured from biopsies of the anterior tibialis muscle and harvested at the time of the infarction (treated group, n=18). Left ventricular function was investigated by ultrasound one month after the transplantation. The baseline value of the ejection fraction was similar in the control (24±2%) and treated (28±1%) animals (p=0.11). One month after the transplantation, the values of ejection fractions had increased in both groups and were 32±2% in the control group and 38±2% in the treated group. However, there was a marked increase in the treated group (p<0.001 versus baseline) compared to the control group (p=0.004 versus baseline), the ejection fraction being significantly higher in the treated rats (p=0.04 versus the control group). Analysis of the volume data showed that the functional improvement produced by transplanting cells of muscular origin was related essentially to an increase in contractility rather than to any change in the left ventricle.

These findings show that there is an additive effect between the transplantation of cells of muscular origin and treatment with ACE inhibitors.

### C. CLINICAL TRIALS IN MAN OF TRANSPLANTING CELLS OF MUSCULAR ORIGIN IN ORDER TO RECONSTITUTE MYOCARDIAL TISSUE

Clinical trial of the transplantation of cells of muscular origin prepared by the method according to the invention have been carried out in human subjects with a view to treating heart failure.

C.1. Methods

The trials were carried out in 6 patients. At the time they were included they were between 18 and 75 years of age. These patients all had clinical indications for aorto-coronary bypass, with possible surgical revascularization. Their global left ventricular ejection fraction (measured by ultrasound and/or angiograph and/or isotopes) was less than or equal to 35%.

They all had a history of transmural myocardial infarction. Finally, the patients presented with segmental hypokinesis or akinesis of the contiguous segments or more extensively (other than an aneurysm) connected to the infarction. The residual metabolic activity in this zone was less than 50%, detected by positron emission tomography (PET), and there was no kinetic increase during ultrasound in response to low dose dobutamine (10 gamma/kg/min).

Ten to eighteen grams of autologous tissue were taken from the patient's vastus lateralis. The incision was performed just at the level of the vastus lateralis. The protocol for culturing the cells and their expansion is as described in part A of the present text.

The cell culture for injection was then placed in a stainless-steel dish and aspirated into a 1-ml syringe. A coronary shunt was first set up with extra-corporeal circulation in the usual manner. After assessing the extent of the infarction and identifying the edges of the necrotic zone, a cell suspension of about 650 to 1200 million cells (1.5×10⁸ cells/ml) was injected into and around the infarcted zone using the 1-ml syringe. Several injections were required to apply all the cells this stage of the operation was carried out with an extra-corporeal circulation and clamping.

C.2. Assessment Methods and Functional Outcome

These trials have shown that this method is feasible and safe for human use. Furthermore, the functional tests were carried out by measuring left ventricular function (segmental and global) and ventricular reshaping, coupled with determination of the cellular metabolic activity. These determinations were carried out using ultrasound methods, such as conventional Doppler echocardiography (measurement of the diameters, volumes, and left ventricular ejection fractions), tissue Doppler of the infarcted zone and dobutamine echocardiography to test for ischemia and viability. The measurements were also obtained using isotope methods, such as positron emission tomography (PET) (uptake of deoxyfluoroglucose in the infarcted zone).

These determinations were carried out before surgery and post surgery after 1 month±8 days and 3 months±8 days.

The results obtained after the patient had received a transplantation of myoblasts are reported below.

The patient was a 72-year old man, admitted with heart failure (NYHA class III) following an extensive infarction of the lower myocardium, which had failed to respond to treatment, because beta-blockers and ACE inhibitors had to be withdrawn when they proved unacceptable. The extracardiac assessment also identified moderate renal failure (creatinine: 200 mmol/L) and bilateral carotid occlusion with no functional repercussions visible on the intracranial Doppler and therefore not calling for any separate vascular intervention.

The ultrasound scan showned that the left ventricular ejection fraction was 20% with extensive akinesis of the lower wall and severe antero-lateral hypokinesis. The lack of viability in the lower wall was demonstrated by the persistence of the akinesis after administration of a low dose of dobutamine. In contrast, the antero-lateral wall displayed a two-phase response to dobutamine (low dose and then high dose), demonstrating viability and ischemia. These findings were confirmed by ₂fluoro¹⁸deoxyglucose (FDG) positron emission tomography (PET), which showed that there was absolutely no viability in the lower wall, but some in the anterior and lateral parts of the left ventricle.

The coronary angiography showed complete, proximal occlusion of the anterior interventricular artery, with delayed opacification of the distal bed via the homolateral collaterals, a tight stenosis of a high diagonal branch and a proximal occlusion of the right coronary artery. The right coronary artery displayed only insignificant irregularities.

In summary, therefore, this patient presented with:
(1) severe impairment of left ventricular function,
(2) an akinetic and metabolically non-viable infarction scar,
(3) an elective indication for a by-pass to arteries other than those affected by the infarct.

Under local anesthesia, a fragment of the vastus lateralis was taken from the patient via a short incision (5 cm). The myoblasts are produced according to the method of the invention described in part A. The number of cells was multiplied by means of several expansions in multitray dishes, making it possible, within two weeks, to obtain 800×10⁶ cells, of which 65% are CD56+ cells. The proportion of viable cells was over 96%.

Two weeks after the biopsy, the patient was rehospitalized in the cardiac surgery department for his by-pass. In view of his hemodynamic vulnerability after the induction of anesthesia (cardiac output 1.5 L/min with a venous oxygen saturation of 55%), counter pulsation was introduced prophylactically by means of an intra-aortic balloon. The anterior diagonal and interventricular arteries were by-passed using a saphenous vein and left internal mammary artery transplant, respectively, with an extracorporeal circulation and continuous, retrograde cardioplegia without cooling. After carrying out coronary anastomosis, the infarcted zone was easily identified. Thirty-three injections of cells, suspended in 5 ml of albumin, were administered into and around the whitish necrotic foci using a 27 G right-angled needle specially designed to make it possible to set up sub-epicardial gutters with a virtually phlyctena-like appearance. The duration of aortic clamping was 56 min, 16 of which were occupied by the injections of the cells. There was no bleeding from the sites of injection, and the extra-corporeal circulation could be discontinued without any difficulty. The patient was taken off the counter pulsation, and pharmacological back-up with dobutamine during the first three days after surgery and was discharged on day 8 after straightforward sequelae.

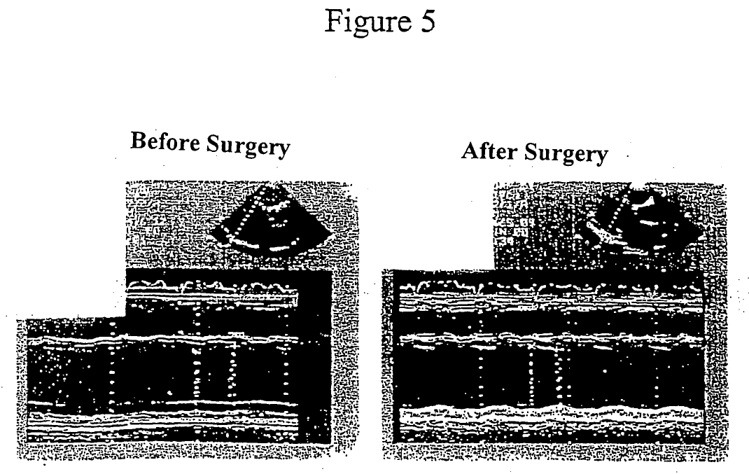

Eight months later, his clinical condition had improved and he is currently NYHA class II, although his medical treatment remains unaltered. A 24-hour Holter did not detect any arrhythmia. Studies of the 4 echocardiographs carried out each month after surgery showed that the left ventricular ejection fraction had increased by 30%. As FIG. 5 shows, segmental contractility was detected not only in the anterior wall, but also in the posterior infarcted and transplanted zone which contracted (the percentage of systolic thickening rose from being virtually imperceptible values before surgery to 40%).

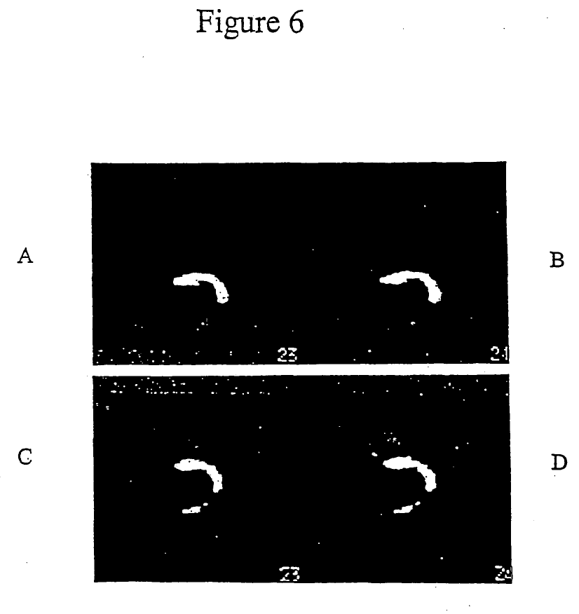

One new fact, this contractility improved further under dobutamine. In addition, tissue Doppler imaging showed the onset of a gradient of the systolic transmyocardial velocity. A fresh FDG PET clearly showed the uptake of the trace by the lower wall, with an activity ratio between the wall and the septum (taken as the control), which rose from 0.5 before the operation to 0.7, which reflects fresh metabolic activity in the infarcted zone that had no viability before surgery (FIG. 6). This last observation cannot have been influenced by the concomitant myocardial revascularization and, combined with the ultrasound data, suggests that the functional improvement in the infarcted zone was indeed related to the presence of the implanted myoblasts.

Taken as a whole, these findings show that the function of the infarcted zone had been improved by the transplantation of myoblasts prepared by the method of the invention.

The injection of autologous muscle cells, prepared according to the method, was also carried out in another 5 patients. The following tables summarize the clinical follow-up data in 4 out of 5 patients (designated as MYO3, MYO4, MYO5 and MYO6 respectively). The clinical follow-up of the sixth patient is in progress.

| | |
|---|---|
| Name of the patient | D. MYO3 |
| Age | 62 |
| Sex | M |
| Type of disorder | Post-ischemic heart failure, labile angina |
| Ejection fraction | 25% |
| NYHA classification stage | III |
| PET-scan | Non-viable zone detected |
| Initial ultrasound | Anterior akinesis, apical dyskinesis, lateral |
| | hypokinesis |
| Virological assessment | Negative |
| Duration of culture | 18 D |
| Cells: number, characteristics | 922 × 10⁶. CD56+: 91%; CD15+: 6%; |
| | desmin+: 65%; Viability: 98%; Myotube |
| | formation (functional test): + |
| Site of injection | Anterior: 900 × 10⁶ cells injected |
| Infectious complications | No post-operative infectious |
| | complications |
| Functional assessment: | |
| NYHA stage | III -> II |
| Ejection fraction | 25% -> 35% |
| Segmental contractility | Increased (±) |
| Change in segmental viability | Improved (+) |

| | |
|---|---|
| Name of the patient | E. MYO4 |
| Age | 67 |
| Sex | M |
| Type of disorder | Post-ischemic heart failure |
| Ejection fraction | 31% |
| NYHA classification stage | III |
| PET-scan | Non-viable zone detected |
| Initial ultrasound | Apical akinesis, posterior akinesis, lateral |
| | hypokinesis |
| Virological assessment | Negative |
| Duration of culture | 20 D |
| Cells: number, characteristics | 657 × 10⁶. CD56+: 97%; CD15+: 15%; |
| | desmin+: 58%; HLA Classel: 94%; |
| | Viability: 98%; Myotube formation |
| | (functional test): + |
| Site of injection | Posterior: 620 × 10⁶ cells injected |
| Infectious complications | No post-operative infectious |
| | complications |
| Functional assessment: | |
| NYHA stage | III -> II |
| Ejection fraction | 31% -> 50% |
| Segmental contractility | Increased (±) |
| Change in segmental viability | Improved (+) |

To summarize, these trials have shown that it is possible to obtain the number of cells required within a period of 2 to 4 weeks. They have also shown that taking the tissue, the preparation and transplantation of human autologous muscle cells could be carried out without pre-, per- or post-surgical difficulties or complications.

Finally, these trials also detected a clinical improvement, an increase in the regional contractility (ultrasound) combined with an increase in the area of the viable zone (PET-scan) in these patients.

In conclusion, the studies reported in parts B and C have yielded the following data:
a) they show that the transplantation of cells of muscular origin significantly improves left ventricular function following myocardial infarction,
b) that the improvement clearly depends on the number of cells injected,
c) this transplantation potentiates a pharmacological treatment, notably with an ACE inhibitor.

d) And, finally, that the method according to the invention is suitable for the treatment of heart failure in man.

| | |
|---|---|
| Name of the patient | F. MYO5 |
| Age | 39 |
| Sex | M |
| Type of disorder | Post-ischemic heart failure |
| Ejection fraction | 22% |
| NYHA classification stage | III |
| PET-scan | Non-viable zone detected |
| Initial ultrasound | Apical akinesis, posterior akinesis, |
| | anterior hypokinesis, lateral hypokinesis |
| Virological assessment | Negative |
| Duration of culture | 14 D |
| Cells: number, characteristics | 993 × 10⁶. CD56+: 95%; CD15+: 4%; |
| | desmin+: 78%; HLA Classel: 94%; |
| | Viability: 98%; Myotube formation |
| | (functional test): + |
| Site of injection | Postero-lateral: 950 × 10⁶ cells injected |
| Infectious complications | No post-operative infectious complications |
| Functional assessment: | |
| NYHA stage | III -> II |
| Ejection fraction | 22% -> 36% |
| Segmental contractility | Increased (±) |
| Change in segmental viability | Improved (+) |

| | |
|---|---|
| Name of the patient | G. MYO 6 |
| Age | 55 |
| Sex | M |
| Type of disorder | Post-ischemic heart failure |
| Ejection fraction | 34% |
| NYHA classification stage | IV |
| PET-scan | Non-viable zone detected |
| Initial ultrasound | Anterior akinesis, lateral akinesis, apical |
| | dyskinesis, septal hypokinesis |
| Virological assessment | Negative |
| Duration of culture | 16 D |
| Cells: number, characteristics | 1210 × 10⁶. CD56+: 85%; CD15+: 10%; |
| | desmin+: 85%; HLA Classel: 94%; |
| | Viability: 97%; Myotube formation |
| | (functional test): + |
| Site of injection | Anterior: 1150 × 10⁶ cells injected |
| Infectious complications | Before the injection, the patient was in a |
| | state of cardiovascular shock and on |
| | adrenaline and noradrenaline. No |
| | post-operative infectious |
| | complications (24 h) after the injection. |
| Functional assessment: | Patient died on 28/APR/01. Cause of death |
| NYHA stage | not attributable to injecting the cells |
| Ejection fraction | (probably cardiovascular shock |
| Segmental contractility | followed by mesenteric ischemia) |
| Change in segmental viability | Not applicable |

1. Method for obtaining a cell population consisting of a dominant cell type from a muscle tissue biopsy for the preparation of a cell therapy product suitable for human administration, the said method including the following steps:
a) taking and shredding of a muscle biopsy specimen,
b) enzymatic dissociation of the muscle fibers and cells, and separation of the individual cells by filtration,
c) culture of cells derived from muscular tissue thus obtained in an adherent cell culture reactor in the presence of growth medium and/or differentiation medium followed, as appropriate by one or more expansion phases,
d) identification of the cell types present at the various stages of the culture by analysis of specific cell markers,
e) selection of the stage of culture during which the required cell type constitutes the dominant proportion of the cell population,
f) harvesting of a population of cells at the culture stage chosen in e),
g) if appropriate, freezing of the cells removed at the stage chosen for the preparation of the cell therapy product.

2. Method according to claim 1, in which only the non-adhering cells are harvested at the culture stage chosen on the basis of their identification in step d).

3. Method according to claim 1 or 2 in which the cell types are identified by analysis of the surface antigens and/or cell markers.

4. Method according to claim 3, in which the antigens or cell markers analysed are chosen from amongst the markers CD10, CD13, CD15, CD34, CD44, CD56, CD117, Class-1 HLA and desmine.

5. Method according to claim 4, in which the antigens or cell markers analysed are the CD34, CD15, CD56 or HLA Class 1 markers.

6. Method according to any of claims 1 to 5, in which the cell culture steps are carried out in a completely closed apparatus in simple, double or multi-level trays.

7. Method according to any of claims 1 to 6, in which the cell culture medium used for the differentiation kinetics is a medium appropriate for differentiating between myoblastic, endothelial, smooth muscle and myofibrillar cells and bone, adipose and cartilaginous cells.

8. Method according to claim 7, in which the appropriate medium for differentiating into myoblastic, endothelial, smooth muscle and myofibroblast cells contains a glucocorticoid and bFGF.

9. Method according to claim 8, in which the medium D-valine has been substituted for L-valine.

10. Method according to claim 7, in which the medium appropriate for differentiating the bone tissue cells contains dexamethasone, beta-glycerophosphate, ascorbate and, if appropriate, fetal calf serum.

11. Method according to claim 7, in which the medium appropriate for differentiating the adipose tissue cells contains dexamethasone, isobutyl-methylxanthine and, if appropriate, indomethacin, fetal calf serum and insulin.

12. Method according to claim 7, in which the medium appropriate for differentiating the cartilaginous tissue cells is a serum-free medium containing TGF-beta3.

13. Method for producing a cell population according to any of claims 1 to 12, characterized by the fact that it does not include any steps involving the purification, positive selection or cloning of a specific cell type.

14. Method for producing a cell population with a high degree of purity from a muscle tissue biopsy for a cell therapy product suitable for human administration, involving:
a) the implementation of a method for producing a cell population according to any of claims 1 to 13,
b) the selection of cells of a target cell type within the cell population obtained in step a)
c) one or more expansion phases of the cells selected in b),
d) if appropriate, the freezing of the cell population obtained.

15. Method for producing a cell population including at least one dominant cell type expressing the CD34 marker, the said method involving the implementation of the method according to any of claims 1 to 13 and in which the culture stage chosen for harvesting the cells is between the D0 stage and the D5 stage.

16. Method for producing a cell population containing one cell type expressing the CD117 marker, the said method involving the implementation of the method according to any of claims 1 to 13 and in which the non-adhering cells are harvested at the culture stage chosen between the D0 stage and the D1 stage.

17. Method for producing a cell population including at least one dominant cell type expressing the CD15 marker, the said method involving the implementation of the method according to claim 8 or 9 and characterized by the fact that the cells are harvested at the culture stage during which the dominant cell type expressed the CD15 marker.

18. Method for producing a cell population including at least one dominant cell type with the characteristics of myoblastic cells or expressing the CD56 marker, by implementing the method according to claim 8 or 9, and in which the culture stage chosen for harvesting the cells occurs after at least 5 days in culture.

19. Method according to claim 18, characterized by the fact that the characteristics of the myoblastic cells are determined by analysis of the CD56 markers, and possibly of the CD10, CD13, CD44, HLA Class 1 and desmine markers.

20. Population of cells in which the dominant cell type expresses the CD34 marker and which is characterized by the fact that it can be produced by implementing the method according to claim 15.

21. Population of cells in which the dominant cell type expresses the CD15 marker and which is characterized by the fact that it can be produced by implementing the method according to claim 17.

22. Population of cells in which a cell type is characterized by the absence of the CD15 and CD56 markers and which is characterized by the fact that it can be produced by implementing the method according to claim 17 or 18.

23. Population of cells in which the cell type has the characteristics of myoblastic cells characterized by the fact that it is produced by implementing the method according to claim 18 or 19.

24. Use of a population of cells obtained according to any of claims 1 to 19 in the preparation of a cell therapy product as a platform for the delivery of a biologically active product.

25. Use of a population of cells obtained according to claim 20 or 21 in the preparation of a cell therapy product for the reconstitution of hematological or immunological system or of bone, adipose, cartilaginous, muscular or vascular tissues.

26. Use of a population of cells obtained according to claim 21 to 23 in the preparation of a cell therapy product for treating muscular and/or joint and/or osteo-tendinous lesions of traumatic origin
for reconstituting of skeletal, cardiac and smooth muscle tissues, for repairing cardiac muscle tissue,
for treating post-ischemic cardiac failure,
for treating heart disease of genetic, viral, iatrogenic, infectious or parasitic origin,
for treating innate or acquired muscular dystrophy for the treatment of vascular diseases.

27. Use of a population of cells obtained according to claim 23 in the preparation of a cell therapy product for treating post-ischemic heart failure.

28. Use of a population of cells obtained according to any of claims 20 to 23 in the preparation of a cell therapy product to potentiate the pharmacological treatment of heart failure.
